(19) United States
(12) Patent Application Publication
(10) Pub. No.: US 2024/0066223 A1
(43) Pub. Date: Feb. 29, 2024

(54) Automatic Suspension and Resumption of Medicament Delivery

(71) Applicant: Dexcom, Inc., San Diego, CA (US)

(72) Inventor: Nathanael R. Paul, San Diego, CA (US)

(73) Assignee: Dexcom, Inc., San Diego, CA (US)

(21) Appl. No.: 18/361,827

(22) Filed: Jul. 29, 2023

Related U.S. Application Data
(60) Provisional application No. 63/400,648, filed on Aug. 24, 2022.

(51) Int. Cl. A61M 5/172; A61M 5/142

(52) U.S. Cl. CPC A61M 5/1723; A61M 5/14248; A61M 2205/3553; A61M 2205/50; A61M 2230/201; A61M 2230/63

(57) ABSTRACT

Automatic suspension and resumption of medicament delivery is described. In one or more implementations, a request to suspend delivery of a medicament to a user is received. A medicament delivery system is controlled to suspend delivery of the medicament to the user, and to automatically resume medicament delivery to the user after a suspension time period. In one or more implementations, a medicament delivery system is controlled to suspend delivery of a medicament to a user during performance of an activity, and to automatically resume delivery of the medicament to the user after performance of the activity is completed. In one or more implementations, a medicament delivery system is controlled to suspend medicament delivery to a user at a first time based on a location of the user, and to resume medicament delivery to the user at a second time based on a subsequent location of the user.

### RELATED APPLICATIONS

This application claims priority under 35 U.S.C. § 119(e) to U.S. Provisional Patent Application No. 63/400,648, filed Aug. 24, 2022, and titled “Automatic Suspension and Resumption of Medicament Delivery,” the entire disclosure of which is hereby incorporated by reference.

### BACKGROUND

Diabetes is a metabolic condition affecting hundreds of millions of people. For these people, monitoring blood glucose levels and regulating those levels to be within an acceptable range is important not only to mitigate long-term issues such as heart disease and vision loss, but also to avoid the effects of hyperglycemia and hypoglycemia. Maintaining blood glucose levels within an acceptable range can be challenging, and in various cases regulating those levels involves administering a medicament (e.g., insulin) throughout the day using a medicament delivery device.

### SUMMARY

To overcome these problems, automatic suspension and resumption of medicament delivery is leveraged. In one or more implementations, a request to suspend delivery of a medicament to a user is received, and a medicament delivery system is controlled to suspend delivery of the medicament to the user. The medicament delivery system is controlled to automatically resume medicament delivery to the user after a suspension time period. In one or more implementations, a medicament delivery system is controlled to suspend delivery of a medicament to a user during performance of an activity, and to automatically resume delivery of the medicament to the user after performance of the activity is completed. In one or more implementations, a medicament delivery system is controlled to suspend delivery of a medicament to a user at a first time based on a location of the user, and to resume delivery of the medicament to the user at a second time based on a subsequent location of the user.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

FIG. 1 is an illustration of an environment in an example implementation that is operable to employ automatic suspension and resumption of medicament delivery.

FIG. 2 depicts an example of an implementation of an analyte monitoring device in greater detail.

FIG. 3 depicts an example implementation of a medicament delivery system in greater detail.

FIG. 4 depicts an example of a system that suspends and resumes delivery of a medicament based on a request.

FIG. 5 depicts an example of a system that suspends delivery of a medicament automatically based on sensor data.

FIG. 6 depicts an example of a system that resumes delivery of a medicament automatically based on sensor data.

FIG. 7 depicts an example of a system that suspends delivery of a medicament automatically based on a location of a user determined using sensor data.

FIG. 8 depicts an example of a system that resumes delivery of a medicament automatically based on a subsequent location of a user determined using sensor data.

FIG. 9 depicts an example of a user interface displaying a delivery suspension notification.

FIG. 10 depicts an example of a user interface displaying a delivery resumption notification.

FIG. 11 depicts an example of a first combination of devices for implementing automatic suspension and resumption of medicament delivery.

FIG. 12 depicts an example of a second combination of devices for implementing automatic suspension and resumption of medicament delivery.

FIG. 13 depicts a procedure in an example implementation of automatic suspension and resumption of medicament delivery after a suspension time period.

FIG. 14 depicts a procedure in an example implementation of automatic suspension and resumption of medicament delivery based on an activity.

FIG. 15 depicts a procedure in an example implementation of automatic suspension and resumption of medicament delivery based on a location of the user.

FIG. 16 illustrates an example of a system generally at that includes an example of a computing device that is representative of one or more computing systems and/or devices that may implement the various techniques described herein.

### DETAILED DESCRIPTION

Overview

Maintaining analyte levels (e.g., blood glucose levels) within an acceptable range can be challenging, and in various cases regulating those levels involves administering a medicament (e.g., insulin) throughout the day using a medicament delivery system (e.g., an insulin pump). As part of controlling analyte levels within an acceptable range, conventional medicament delivery systems may provide the capability to manually suspend and resume medicament delivery. For example, a user can manually send a first command to a conventional insulin pump to suspend insulin delivery, e.g., by providing user input to an application displayed on a user device, removing the insulin pump, and/or providing user input to the insulin pump itself. Then, at a later time, the user can manually send a second command to the insulin pump to resume insulin delivery.

A user may decide to manually suspend medicament delivery for a variety of different reasons using conventional systems. In a first example, a user may decide to manually suspend insulin delivery during exercise because the performance of exercise may drastically decrease the user's blood glucose levels. Thus, in this example, the user may manually suspend insulin delivery during exercise to prevent the combination of insulin delivery and exercise from causing the user's blood glucose levels to decrease below a target range causing the person to experience a hypoglycemic event. Similarly, in a second example, a user may decide to manually suspend insulin delivery (e.g., by removing the insulin delivery device) during bathing or while playing sport. However, in both these examples, conventional systems do not automatically resume insulin delivery, but instead require the user to manually resume insulin delivery. In many cases, however, the user may forget to manually resume insulin delivery which may result in a dangerous health condition for the user, e.g., without insulin delivery enabled the user's blood glucose levels may subsequently increase above a target range associated with hyperglycemia.

To solve these problems, automatic suspension and resumption of medicament delivery is leveraged. In accordance with the described techniques, a delivery device control system is configured to provide automatic suspension and/or resumption of medicament delivery in a way that eliminates or reduces user interaction involved with manually suspending and resuming insulin delivery. In one or more implementations, the medicament delivery system can both automatically suspend and automatically resume insulin delivery based on sensor data indicating a location of the user and/or an activity being performed by the user. For example, the delivery device control system can suspend and/or resume medicament delivery based on a location of a user determined from sensor data. At a first time, for example, the delivery device control system determines or predicts the presence of the user at a first location and, based on the determined or predicted presence of the user at the first location, the delivery device control system causes medicament delivery to be suspended, e.g., by instructing a medicament delivery system (e.g., an insulin pump) to suspend delivery of a medicament to the user. Then, at a subsequent second time, the delivery device control system determines or predicts the presence of the user at a second, different location (or that the user leaves the first location). Based on the determined or predicted presence of the user at the second location (or that the user leaves the first location), the delivery device control system automatically (without user input) causes the medicament delivery to be resumed, e.g., by instructing the medicament delivery system to resume the suspended delivery of the medicament to the user.

Alternatively or in addition, the delivery device control system suspends and/or resumes medicament delivery based sensor data indicating an activity of the user, e.g., an activity performed by the user wearing the medicament delivery system. At a first time, for example, the delivery device control system determines or predicts that the user is performing an activity and, based on the determined or predicted activity causes medicament delivery to be suspended, e.g., by instructing the medicament delivery system to suspend delivery of a medicament to the user. Then, at a subsequent second time, the delivery device control system determines or predicts that the user ceases performing the activity (or that the user performs a different activity). Based on determining or predicting that the user no longer performs the activity (or that the user performs a different activity), the delivery device control system automatically (without user input) causes the medicament delivery to be resumed, e.g., by instructing the medicament delivery system to resume the suspended delivery of the medicament to the person. In at least one variation, the delivery device control system determines an activity that a user performs, at least in part, based on also determining a location of the user. For example, the location of the user (e.g., at the gym) can be used by the delivery device control system to infer an activity (e.g., exercise). However, in different variations, the delivery device control system determines an activity that the user performs without also determining a location of the user.

In one or more implementations, the delivery device control system provides a temporary suspend feature which temporarily suspends medicament delivery for a suspension time period, and then automatically resumes medicament delivery after the suspension time period has lapsed. In this implementation, rather than determine when to suspend delivery without user input, the delivery device control system may suspend delivery based on receiving user input requesting to suspend delivery of the medicament. Such a request may be received, for example, via the medicament delivery system responsive to a user selecting a graphical control displayed via a display device of the medicament delivery system or selecting a physical control (e.g., a button) of the medicament delivery system. In at least one variation, the user also specifies the suspension time period, i.e., how long the delivery device control system is to suspend delivery of the medicament before causing delivery to be resumed. Alternatively, the user does not specify the suspension time period as part of the request, and the delivery device control system determines the suspension time period without the user inputting it. In one or more variations, the delivery device control system causes delivery of the medicament to be resumed automatically (without user input) responsive to determining that the suspension time period has lapsed. In other words, after the suspension time period lapses, the delivery device control system causes delivery of the medicament to be resumed without receiving a user input proximate the time when the delivery is resumed—the delivery device control system resumes delivery based on a maintained value specifying the suspension time period.

Thus, by suspending and/or resuming medicament delivery automatically and without user input, the described techniques reduce or eliminate the dangerous health conditions caused by conventional medicament delivery systems which require the user to both manually suspend and manually resume insulin delivery.

In some aspects, the techniques described herein relate to a system including: one or more sensors; a medicament delivery system configured to deliver a medicament to a user; and a delivery device control system configured to: control the medicament delivery system to suspend delivery of the medicament to the user responsive to a request initiated by the user, and to automatically resume delivery of the medicament to the user after a suspension time period has lapsed; and control the medicament delivery system to automatically suspend delivery of the medicament to the user based on first sensor data obtained from the one or more sensors, and to automatically resume delivery of the medicament to the user based on second sensor data obtained from the one or more sensors.

In some aspects, the techniques described herein relate to a system, wherein the delivery device control system is further configured to control the medicament delivery system to automatically suspend delivery of the medicament to the user based on the first sensor data indicating performance of an activity by the user, and to automatically resume delivery of the medicament to the user based on the second sensor data indicating that performance of the activity is completed.

In some aspects, the techniques described herein relate to a system, wherein the delivery device control system is further configured to control the medicament delivery system to automatically suspend delivery of the medicament to the user based on the first sensor data indicating that the user is at a location, and to automatically resume delivery of the medicament to the user based on second sensor data indicating that the user is at a subsequent location.

In some aspects, the techniques described herein relate to a system, further including an analyte monitoring device that is connected to the medicament delivery system to form a closed loop system.

In some aspects, the techniques described herein relate to a system, wherein the analyte monitoring device includes a wearable glucose monitoring device, and wherein the medicament delivery system includes a wearable insulin pump.

In some aspects, the techniques described herein relate to a system, wherein the delivery device control system is implemented at a computing device that is wirelessly coupled to the medicament delivery system and the analyte monitoring device.

In some aspects, the techniques described herein relate to a system, wherein the delivery device control system is implemented at the analyte monitoring device.

In some aspects, the techniques described herein relate to a system, wherein the delivery device control system is implemented at the medicament delivery system.

In some aspects, the techniques described herein relate to a method including: receiving a request to suspend delivery of a medicament to a user; controlling a medicament delivery system to suspend delivery of the medicament to the user; and controlling the medicament delivery system to automatically resume medicament delivery to the user after a suspension time period.

In some aspects, the techniques described herein relate to a method, wherein the medicament delivery system automatically resumes medicament delivery to the user after the suspension time period has lapsed without user interaction.

In some aspects, the techniques described herein relate to a method, wherein the medicament delivery system is connected to an analyte monitoring device and a computing device to form a closed loop system.

In some aspects, the techniques described herein relate to a method, wherein the request to suspend delivery of the medicament is received responsive to user input to the medicament delivery system.

In some aspects, the techniques described herein relate to a method, wherein the request to suspend delivery of the medicament is received responsive to user input to a user interface displayed at the computing device.

In some aspects, the techniques described herein relate to a method, wherein the request to suspend delivery of the medicament to the user includes an indication of the suspension time period.

In some aspects, the techniques described herein relate to a method, wherein the suspension time period corresponds to a predetermined period of time.

In some aspects, the techniques described herein relate to a method, wherein the medicament includes insulin.

In some aspects, the techniques described herein relate to a method including: obtaining sensor data from one or more sensors; predicting, based on the sensor data, an activity that a user will perform; controlling a medicament delivery system to suspend delivery of a medicament to the user during performance of the activity; and controlling the medicament delivery system to automatically resume delivery of the medicament to the user after performance of the activity is completed.

In some aspects, the techniques described herein relate to a method, further including detecting that performance of the activity is completed based on additional sensor data obtained from the one or more sensors.

In some aspects, the techniques described herein relate to a method, wherein the activity includes exercise, sleeping, or bathing.

In some aspects, the techniques described herein relate to a method, further including detecting, based on the sensor data, a location of a user, wherein the predicting further includes predicting the activity that the user will perform based on the location of the user.

In some aspects, the techniques described herein relate to a method, wherein detecting the location of the user includes detecting the location of the user based on first sensor data, and confirming the location of the user based on second sensor data.

In some aspects, the techniques described herein relate to a method, wherein detecting the location of the user includes: detecting at least two candidate locations of the user based on first sensor data; and selecting one of the at least two candidate locations as the location of the user based on second sensor data.

In some aspects, the techniques described herein relate to a method, wherein the predicting includes predicting, based on the sensor data, a time at which the activity will begin.

In some aspects, the techniques described herein relate to a method, further including controlling the medicament delivery system to administer a bolus dose of the medicament to the predicted time at which the activity will begin.

In some aspects, the techniques described herein relate to a method, wherein the bolus dose of the medicament includes a bolus dose of insulin.

In some aspects, the techniques described herein relate to a method, further including: determining a current glucose measurement of the user based on a glucose monitor worn by the user; and determining the bolus dose of insulin based on the current glucose measurement of the user.

In some aspects, the techniques described herein relate to a method including: controlling a medicament delivery system to suspend medicament delivery to a user at a first time based on a location of the user; controlling the medicament delivery system to resume medicament delivery to the user at a second time based on a subsequent location of the user; and responsive to resuming medicament delivery, causing the medicament delivery system to deliver a dose of medicament to the user.

In some aspects, the techniques described herein relate to a method, wherein the controlling the medicament delivery system to suspend and resume medicament delivery includes controlling the medicament delivery system to suspend and resume medicament delivery automatically without user input.

In some aspects, the techniques described herein relate to a method, wherein the controlling the medicament delivery system to suspend and resume medicament delivery to the user is not based on analyte data.

In some aspects, the techniques described herein relate to a method, wherein the dose of medicament delivered to the user is based at least in part on analyte data.

In some aspects, the techniques described herein relate to a method, wherein the dose of medicament delivered to the user is based at least in part on a time interval between the first time and the second time.

In some aspects, the techniques described herein relate to a method, wherein the dose of medicament delivered to the user is based at least in part on an activity the user performed between the first time and the second time.

In some aspects, the techniques described herein relate to a method, wherein the activity is predicted based at least in part on the location of the user.

In the following discussion, an exemplary environment is first described that may employ the techniques described herein. Examples of implementation details and procedures are then described which may be performed in the exemplary environment as well as other environments. Performance of the exemplary procedures is not limited to the exemplary environment and the exemplary environment is not limited to performance of the exemplary procedures.

### Example of an Environment

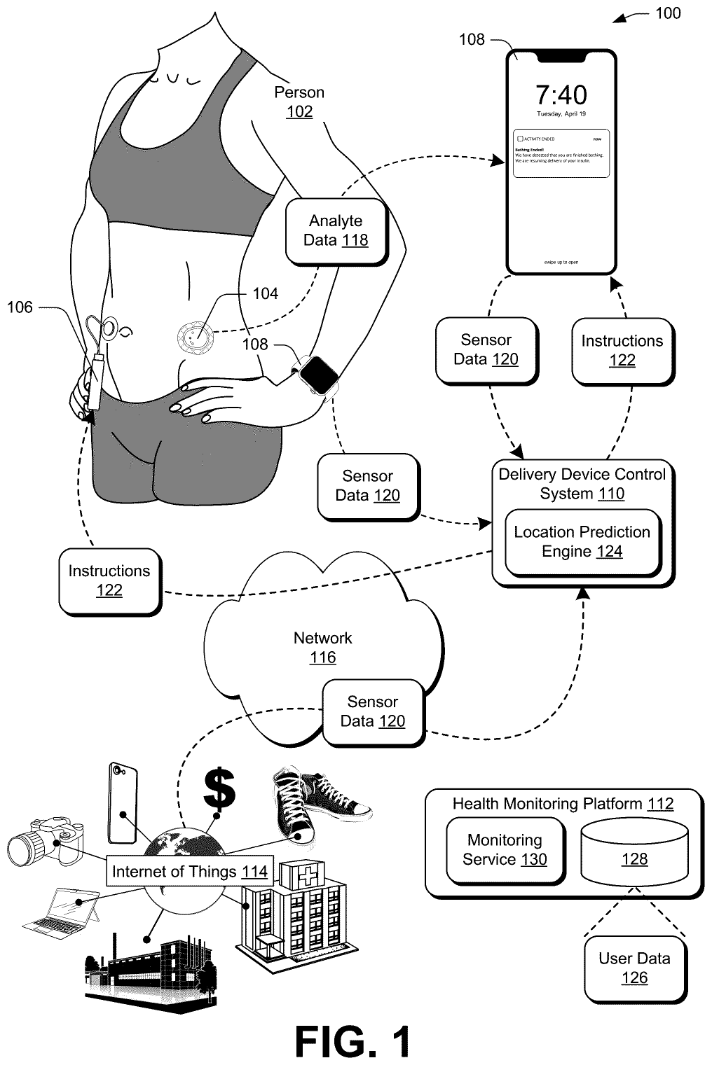

FIG. 1 is an illustration of an environment 100 in an example implementation that is operable to employ automatic suspension and resumption of medicament delivery. The illustrated environment 100 includes person 102, who is depicted wearing analyte monitoring device 104 and medicament delivery system 106. The illustrated environment 100 also includes example computing devices 108, delivery device control system 110, health monitoring platform 112, and Internet of Things (IoT 114). The analyte monitoring device 104, the medicament delivery system 106, the example computing devices 108, the delivery device control system 110, the health monitoring platform 112, and the IoT 114 are communicably coupled, such as via network 116.

The analyte monitoring device 104, the medicament delivery system 106, and one or more computing devices 108 (associated with the person 102) may be communicably coupled in various ways, such as by using one or more wireless communication protocols or techniques. By way of example, the analyte monitoring device 104, the medicament delivery system 106, and one or more computing devices 108 may communicate with one another using one or more of radio, cellular, Wi-Fi, Bluetooth (e.g., Bluetooth Low Energy links), near-field communication (NFC), and 5G, to name just a few.

Through such communicative couplings, one or more of the analyte monitoring device 104, the medicament delivery system 106, and one or more of the computing devices 108 may form a closed-loop system in one or more implementations. When implemented as a closed-loop system, the combination of devices is configured to provide automatic suspension and resumption of medicament delivery in a way that eliminates or reduces user interaction involved with removing the medicament delivery system 106 temporarily, such as to bathe or participate in a physical activity, e.g., basketball. Instead, the devices process various information and make various predictions and determinations, such as about a user's location and/or activities the user is performing or will perform based on the location to automatically suspend and resume medicament delivery. In a closed-loop system, the devices can thus control the medicament delivery system 106 to suspend and subsequently resume delivery of a medicament to the person 102 without user interaction, such as without receiving user input to any controls (e.g., of the medicament delivery system 106 or a computing device 108) for explicitly suspending or resuming medicament delivery.

In one or more implementations, the analyte monitoring device 104 is a wearable, such that it is worn by the person 102 while the device performs various operations. Additionally or alternatively, the analyte monitoring device 104 performs one or more operations before or after being worn by the person 102. Broadly, the analyte monitoring device 104 is configured to provide measurements of an analyte of the person 102, e.g., the person 102's glucose. For instance, the analyte monitoring device 104 may be configured with an analyte sensor that detects one or more signals indicative of the analyte in the person 102 and enables generation of analyte measurements or estimations (e.g., estimated glucose values). Those analyte measurements (e.g., glucose measurements) may correspond to or otherwise be packaged for communication to one or more of the computing devices 108 or the medicament delivery system 106 as analyte data 118.

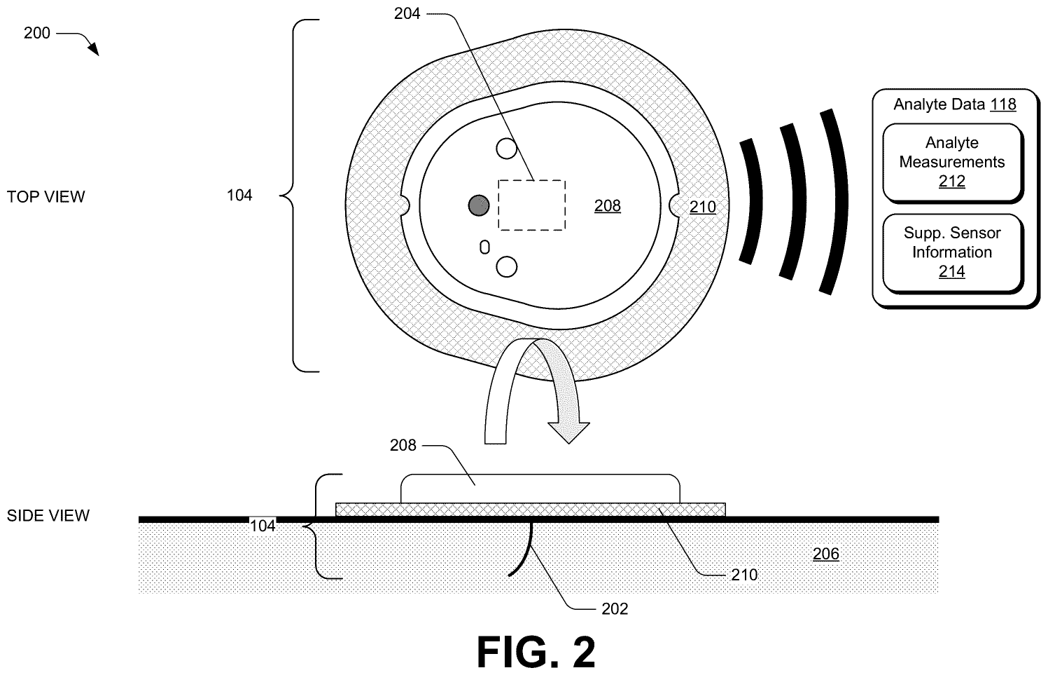

In at least one implementation, the analyte monitoring device 104 is a glucose monitoring system. As an example, the analyte monitoring device 104 may be configured as a continuous glucose monitoring (“CGM”) system, e.g., a wearable CGM system. As used herein, the term “continuous” when used in connection with analyte monitoring may refer to an ability of a device to produce measurements substantially continuously, such that the device may be configured to produce the analyte measurements at regular or irregular intervals of time (e.g., approximately every hour, approximately every 30 minutes, approximately every 5 minutes, and so forth), responsive to establishing a communicative coupling with a different device (e.g., when a computing device 108 establishes a wireless connection with the analyte monitoring device 104 to retrieve one or more of the measurements), and so forth. In other implementations, however, the glucose monitoring device may not be “continuous”, and instead provides glucose measurements when requested. For example, the analyte monitoring device 104 may communicate a current glucose measurement to the computing device 108 responsive to a request from the computing device 108. This request may be initiated in a variety of different ways, such as responsive to user input to a user interface displayed by the computing device 108 (and/or a different device), responsive to placement of the computing device 108 within a threshold proximity to the analyte monitoring device 104, responsive to the computing device 108 making physical contact with the analyte monitoring device 104, responsive to a request from an application implemented at the computing device 108, and so forth. This functionality along with further aspects of the configuration of the analyte monitoring device 104 are discussed in more detail in relation to FIG. 2.

In addition to producing analyte data 118, the analyte monitoring device 104 also transmits the produced analyte data 118, e.g., to the computing device 108. The analyte monitoring device 104 may communicate the data in real-time, e.g., as it is produced using an analyte sensor. Alternatively or in addition, the analyte monitoring device 104 may communicate the data to a computing device 108 at predefined intervals of time. For example, the analyte monitoring device 104 may be configured to communicate the analyte data 118 to the computing device 108 approximately every five minutes. Certainly, an interval at which the analyte data 118 is communicated by the analyte monitoring device 104 may be different from the examples above without departing from the spirit or scope of the described techniques. In other implementations, the analyte data 118 is communicated by the analyte monitoring device 104 when requested. For example, the analyte monitoring device 104 device may communicate a current analyte measurement to the computing device 108 responsive to a request from the computing device 108. This request may be initiated in a variety of different ways, such as responsive to user input to a user interface displayed by the computing device 108 (and/or a different device), responsive to placement of the computing device 108 within a threshold proximity to the analyte monitoring device 104, responsive to the computing device 108 making physical contact with the analyte monitoring device 104, responsive to a request from an application implemented at the computing device, and so forth. The data may be communicated by the analyte monitoring device 104 to a computing device 108 according to other bases in accordance with the described techniques.

Additionally, the computing device 108 may maintain the analyte data 118 and sensor data 120 received from various sources at least temporarily, e.g., in a storage device (not shown) of the computing device 108. The analyte data 118 and the sensor data 120 may also be maintained in such storage of the computing device 108 or storage of a different device along with other associated data, such as with corresponding timestamps and/or identifiers of data packets in which communicated, to name just a few.

As depicted, the described systems may include one or more computing devices 108 in accordance with the described techniques. In one or more scenarios, for instance, the described techniques may be implemented using a computing device 108, such as a mobile phone. Alternatively, the described techniques are implementable using multiple computing devices 108, which in at least one variation includes both a wearable device (e.g., a smart watch, mouthguard, contact lenses, smart glasses, chest strap, ear buds, or headphones, to name just a few) and a mobile phone. In such scenarios, both of these devices may be capable of performing at least some of the same operations, such as to receive the analyte data 118 from the analyte monitoring device 104, receive the sensor data 120 from various sources, generate the sensor data 120 using onboard or associated sensors, communicate data via the network 116 to the delivery device control system 110 and/or the health monitoring platform 112, display information related to the data, display information related to automatic medicament suspension or resumption regulated by the delivery device control system 110, facilitate control of the medicament delivery system 106, and so forth. Alternatively or in addition, different devices may have different capabilities that other devices do not have or that are limited to specified devices through computing instructions.

The delivery device control system 110 controls the medicament delivery system 106 to suspend delivery of one or more medicaments and subsequently resume delivery of the one or more medicaments. The delivery device control system 110 controls the medicament delivery system 106, at least in part, by processing the sensor data 120 and by providing instructions 122, such as by providing the instructions 122 to the medicament delivery system 106, the computing device 108, and/or to another device (e.g., an additional medicament delivery system). In accordance with the described techniques, the delivery device control system 110 makes determinations regarding medicament delivery and causes it to be suspended and/or resumed, at least in part, by leveraging one or more of the computing devices 108, the medicament delivery system 106, the health monitoring platform 112, and the IoT 114.

Although depicted as being separate from the analyte monitoring device 104, the medicament delivery system 106, the computing devices 108, and the health monitoring platform 112, in one or more implementations, at least a portion of the delivery device control system 110 is implemented at one or more of those entities. Additionally, various portions of the delivery device control system 110 are implementable at or otherwise using different combinations of the analyte monitoring device 104, the medicament delivery system 106, the computing devices 108, and the health monitoring platform 112 in accordance with the described techniques.

As discussed above and below, the delivery device control system 110 obtains the sensor data 120 from various sources. For example, the delivery device control system 110 obtains the sensor data 120 from one or more of the medicament delivery system 106, the computing devices 108, the health monitoring platform 112, or the IoT 114. Examples of the sensor data 120 include, but are not limited to, global positioning system (GPS) data, Wi-Fi information (e.g., service set identifiers (SSIDs) of available networks, networks to which the computing device 108 is connected, and/or networks to which the computing device 108 previously connected), Bluetooth Low Energy (BLE) information, data produced using a cellular antenna (e.g., long term evolution (LTE)), microphone data (e.g., sound data), accelerometer data, gyroscope data, magnetometer data, barometer data, ambient or internal temperature data (e.g., produced using temperature sensors), light data (e.g., captured using a device's camera), heart rate data (e.g., produced by a small phone and/or a smart watch), proximity data (e.g., between devices), humidity data, analyte data, and medicament data, to name just a few. It is to be appreciated that this list is not exhaustive, and that the delivery device control system 110 may receive various other data (some of which is discussed above and below), without departing from the spirit or scope of the described techniques.

In the illustrated example, the delivery device control system 110 is depicted including location prediction engine 124. However, the delivery device control system 110 may include more, fewer, or different components without departing from the spirit or scope of the described techniques. Some examples of various configurations of the delivery device control system 110 are discussed below.

Based on the sensor data 120, the location prediction engine 124 detects a location of a user, e.g., the person 102 wearing the medicament delivery system 106. In one or more implementations, the location prediction engine 124 detects the location of the user based on at least two different types of the sensor data 120. For instance, the location prediction engine 124 detects the location of the user based on both GPS data and Wi-Fi information (e.g., an SSID to which the computing device 108 of the user is connected) or based on Wi-Fi information and sound data. In one or more implementations, the location prediction engine 124 uses the first sensor data to detect a location of the user and the second sensor data to confirm the location of the user. Alternatively or in addition, the location prediction engine 124 uses the two types of sensor data to distinguish which of multiple candidate locations corresponds to the actual physical location of the user. For instance, the location prediction engine 124 detects at least two candidate locations of the user based on the first sensor data. Based on the second sensor data, the location prediction engine 124 selects one of the at least two candidate locations as the location of the user (or eliminates all but one of the candidate locations).

In one or more implementations, the delivery device control system 110 suspends and/or resumes medicament delivery based on a location of a user, e.g., the person 102 wearing the medicament delivery system 106. At a first time, for example, the location prediction engine 124 determines or predicts the presence of the user at a first location and, based on the determined or predicted presence of the user at the first location, the delivery device control system 110 causes medicament delivery to be suspended, e.g., by instructing the medicament delivery system 106 to suspend delivery of a medicament to the person 102. Then, at a subsequent second time, the location prediction engine 124 determines or predicts the presence of the user at a second, different location (or that the user leaves the first location). Based on the determined or predicted presence of the user at the second location (or that the user leaves the first location), the delivery device control system 110 automatically (without user input) causes the medicament delivery to be resumed, e.g., by instructing the medicament delivery system 106 to resume the suspended delivery of the medicament to the person 102.

Alternatively or in addition, the delivery device control system 110 suspends and/or resumes medicament delivery based on an activity of the user, e.g., an activity performed by the person 102 wearing the medicament delivery system 106. At a first time, for example, the delivery device control system 110 determines or predicts that the user is performing an activity and, based on the determined or predicted activity causes medicament delivery to be suspended, e.g., by instructing the medicament delivery system 106 to suspend delivery of a medicament to the person 102. Then, at a subsequent second time, the delivery device control system 110 determines or predicts that the user ceases performing the activity (or that the user performs a different activity). Based on determining or predicting that the user no longer performs the activity (or that the user performs a different activity), the delivery device control system 110 automatically (without user input) causes the medicament delivery to be resumed, e.g., by instructing the medicament delivery system 106 to resume the suspended delivery of the medicament to the person 102. In at least one variation, the delivery device control system 110 determines an activity that a user performs, at least in part, based on also determining a location of the user. However, in different variations, the delivery device control system 110 determines an activity that the user performs without also determining a location of the user.

In one or more implementations, the delivery device control system 110 determines when to suspend delivery of the medicament based on the sensor data 120, and not based on the analyte data 118. In other implementations, the delivery device control system 110 determines when to suspend delivery of the medicament based, at least in part, on the analyte data 118. Likewise, the delivery device control system 110 determines when to resume delivery of the medicament, in one or more implementations, based on the sensor data 120 and not based on the analyte data 118. In other implementations, though, the delivery device control system 110 determines when to resume delivery of the medicament based, at least in part on the analyte data 118. Whether the delivery device control system 110 uses the analyte data 118 or not to determine when to resume medicament delivery (and instruct the medicament delivery system 106 accordingly), the delivery device control system 110 may use the analyte data 118 to determine a dose of the medicament to deliver when delivery is resumed.

In at least one implementation, rather than determine when to suspend delivery without user input, the delivery device control system 110 suspends delivery based on receiving user input requesting to suspend delivery of the medicament. In one or more implementations, such a request may be received via the medicament delivery system 106, for instance, responsive to a user selecting a graphical control displayed via a display device of the medicament delivery system 106 or selecting a physical control (e.g., a button) of the medicament delivery system 106. In at least one variation, the user also specifies a suspension time period, i.e., how long the delivery device control system 110 is to suspend delivery of the medicament before causing delivery to be resumed. Alternatively, the user does not specify the suspension time period as part of the request, and the delivery device control system 110 determines the suspension time period without the user inputting it. In one or more variations, the delivery device control system 110 causes delivery of the medicament to be resumed automatically (without user input) responsive to determining that the suspension time period has lapsed. In other words, after the suspension time period lapses, the delivery device control system 110 causes delivery of the medicament to be resumed without receiving a user input proximate the time when the delivery is resumed—the delivery device control system 110 resumes delivery based on a maintained value specifying the suspension time period.

As noted above, the delivery device control system 110 uses sensor data 120 obtained via the IoT 114 in one or more implementations. It is to be appreciated that the IoT 114 represents various sources capable of providing data that describes the person 102 and/or the person 102's activity, such as the person 102's activity as a user of one or more service providers or the person 102's activity within the real world, e.g., at home, in an automobile, at work, in a gym, in a restaurant, or in other stores, to name just a few. By way of example, the IoT 114 may include various devices of the user, e.g., mobile phones, wearable devices, cameras, laptops, and so forth. To this end, the IoT 114 may provide information about interaction of the user with those various devices, e.g., location of those devices, environmental and/or physical conditions at locations where the devices are placed, interaction with web-based applications supported by the devices, interaction with health applications supported by the devices, photos taken, communications with other users, online behavior, and so forth. The IoT 114 may also include various other real-world articles (e.g., shoes, clothing, sporting equipment, appliances, smart-home devices, automobiles, etc.) configured with sensors to provide information describing behavior, such as household items or locations being interacted with (e.g., shower, bathtub, or in bed), steps taken, force of a foot striking the ground, length of stride, temperature of a user (and other physiological measurements), temperature of a user's surroundings, types of food stored in a refrigerator, types of food removed from a refrigerator, driving habits, images of the user at different times of the day, and so forth. Such other real-world articles can also provide sensor data 120 describing location of those articles, user interaction with those articles, environmental and/or physical conditions at locations where the articles are placed, and various other conditions.

In variations, the IoT 114 may also include third parties to the health monitoring platform 112, such as medical providers (e.g., a medical provider of the person 102) and manufacturers (e.g., manufacturers of the medicament delivery system 106 or one or more of the computing devices 108) capable of providing medical and manufacturing data, respectively, that can be leveraged by the delivery device control system 110. Certainly, the IoT 114 may include devices and sensors capable of providing a wealth of data used to determine a location and/or activities of the person 102 without departing from the spirit or scope of the described techniques.

In one or more implementations, the delivery device control system 110 also leverages resources of the health monitoring platform 112 in connection with automatic suspension and resumption of medicament delivery. For instance, the health monitoring platform 112 may be configured to store data, such as the analyte data 118, the sensor data 120 (e.g., data produced by various sensors and data produced based on determinations made using the data produced by the various sensors), the instructions 122, user profile data associated with a user (e.g., the person 102), and/or user profile data associated with one or more other users of a user population (not shown). The environment 100 includes user data 126, which represents a variety of data obtained and stored by the health monitoring platform 112 about one or more of those users. Although stored about one or more users, the user data 126 may be obfuscated using one or more techniques, such that personally identifying information of the users associated with the data can be kept anonymous in various scenarios of using the data. In the illustrated environment 100, the user data 126 is shown stored in the storage device 128. The storage device 128 may represent one or more databases or other storage included as part of or otherwise accessible to the health monitoring platform 112. In accordance with the described techniques, the storage device 128 is capable of storing the user data 126 and various other data.

By way of example, the user data 126 may include any combination of the data discussed just above as well as other data discussed above and below. For instance, the user data 126 may include historical data associated with the one or more users, such as historical analyte data 118, historical sensor data 120, determinations made based on the historical analyte data 118 and/or the historical sensor data 120, user inputs received, and so on. In at least one implementation, such historical data may describe conditions of the users and/or behaviors of the users, including, for instance, activities previously performed by a user at a location, activities previously performed by other users at the location, and attributes of the performed activities, e.g., an activity time, a location type, whether the user provided input to suspend medicament delivery, whether the user provided input to resume medicament delivery, an amount of time medicament delivery was suspended before being resumed, etc.

In one or more implementations, the health monitoring platform 112 includes a monitoring service 130. The monitoring service 130 may be used separately or in connection with the delivery device control system 110. By way of example, the monitoring service 130 may provide one or more web-based health-related services to users via the network 116 and their computing devices 108, e.g., mobile applications. The health monitoring platform 112 may include or have access to various computing resources, such as processing, storage, and virtual resources. Those resources may be useable, for example, to train, maintain, and/or deploy algorithms (e.g., machine learning algorithms), which may generate predictions associated with health monitoring by using the wealth of data collected about the person 102 and users of a user population. Thus, the monitoring service 130 may leverage those resources to execute algorithms and to implement other functionality in order to deliver web-based services to users via their devices.

Notably, one or more such algorithms or functionalities may require an amount of computing resources that exceeds the resources of typical, personal computing devices, e.g., mobile phones, laptops, tablet devices, and wearables, to name just a few. However, the health monitoring platform 112 may include or otherwise have access to at least a threshold amount of resources needed to operate such algorithms and provide such functionality, e.g., cloud storage, server devices, virtualized resources, and so forth. The health monitoring platform 112 may include a variety of resources that the computing devices 108 can leverage via the monitoring service 130 in order to automatically suspend and resume medicament delivery and provide notifications about doing so. In the context of measuring an analyte, e.g., glucose continuously, and obtaining analyte data describing such measurements, consider the following discussion of FIG. 2.

FIG. 2 depicts an example 200 of an implementation of the analyte monitoring device 104 in greater detail. In particular, the illustrated example 200 includes a top view and a corresponding side view of the analyte monitoring device 104. It is to be appreciated that the analyte monitoring device 104 may vary in implementation from the following discussion in various ways without departing from the spirit or scope of the described techniques.

In this example 200, the analyte monitoring device 104 is illustrated to include an analyte sensor 202 (e.g., a glucose sensor) and a sensor module 204. Here, the analyte sensor 202 is depicted in the side view having been inserted subcutaneously into skin 206, e.g., of the person 102. The sensor module 204 is approximated in the top view as a dashed rectangle. The analyte monitoring device 104 also includes a transmitter 208 in the illustrated example 200. Use of the dashed rectangle for the sensor module 204 indicates that it may be housed or otherwise implemented within a housing of the transmitter 208. Antennae and/or other hardware used to enable the transmitter 208 to produce signals for communicating data, e.g., over a wireless connection to the medicament delivery system 106 and/or the computing device 108, may also be housed or otherwise implemented within the housing of the transmitter 208. In this example 200, the analyte monitoring device 104 further includes adhesive pad 210.

In operation, the analyte sensor 202 and the adhesive pad 210 may be assembled to form an application assembly, where the application assembly is configured to be applied to the skin 206 so that the analyte sensor 202 is subcutaneously inserted as depicted. In such scenarios, the transmitter 208 may be attached to the assembly after application to the skin 206 via an attachment mechanism (not shown). Alternatively, the transmitter 208 may be incorporated as part of the application assembly, such that the analyte sensor 202, the adhesive pad 210, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, user-initiated application of the analyte monitoring device 104 with a sensor applicator is nearly painless and does not require the withdrawal of blood. Moreover, the automatic sensor applicator generally enables the person 102 to embed the analyte sensor 202 subcutaneously into the skin 206 without the assistance of a clinician or healthcare provider.

The analyte monitoring device 104 may also be removed by peeling the adhesive pad 210 from the skin 206. It is to be appreciated that the analyte monitoring device 104 and its various components as illustrated are simply one example form factor, and the analyte monitoring device 104 and its components may have different form factors without departing from the spirit or scope of the described techniques.

In operation, the analyte sensor 202 is communicably coupled to the sensor module 204 via at least one communication channel which can be a wireless connection or a wired connection. Communications from the analyte sensor 202 to the sensor module 204 or from the sensor module 204 to the analyte sensor 202 can be implemented actively or passively and these communications can be continuous (e.g., analog) or discrete (e.g., digital).

The analyte sensor 202 may be a device, a molecule, and/or a chemical which changes or causes a change in response to an event which is at least partially independent of the analyte sensor 202. The sensor module 204 is implemented to receive indications of changes to the analyte sensor 202 or caused by the analyte sensor 202. For example, the analyte sensor 202 can include glucose oxidase which reacts with glucose and oxygen to form hydrogen peroxide that is electrochemically detectable by the sensor module 204 which may include an electrode. In this example, the analyte sensor 202 may be configured as or include a glucose sensor configured to detect analytes in blood or interstitial fluid that are indicative of glucose level using one or more measurement techniques. In one or more implementations, the analyte sensor 202 may also be configured to detect analytes in the blood or the interstitial fluid that are indicative of other markers, such as lactate levels, ketones, or ionic potassium, which may improve accuracy in identifying or predicting glucose-based events (e.g., hyperglycemia or hypoglycemia). Additionally or alternatively, the analyte monitoring device 104 may include additional sensors and/or architectures to the analyte sensor 202 to detect those analytes indicative of the other markers.

In another example, the analyte sensor 202 (or an additional sensor of the analyte monitoring device 104—not shown) can include a first and second electrical conductor and the sensor module 204 can electrically detect changes in electric potential across the first and second electrical conductor of the analyte sensor 202. In this example, the sensor module 204 and the analyte sensor 202 are configured as a thermocouple such that the changes in electric potential correspond to temperature changes. In some examples, the sensor module 204 and the analyte sensor 202 are configured to detect a single analyte, e.g., glucose. In other examples, the sensor module 204 and the analyte sensor 202 are configured to use diverse sensing modes to detect multiple analytes, e.g., ionic sodium, ionic potassium, carbon dioxide, and glucose. Alternatively or additionally, the analyte monitoring device 104 includes multiple sensors to detect not only one or more analytes (e.g., ionic sodium, ionic potassium, carbon dioxide, glucose, and insulin) but also one or more environmental conditions (e.g., temperature, moisture, movement). Thus, the sensor module 204 and the analyte sensor 202 (as well as any additional sensors) may detect the presence of one or more analytes, the absence of one or more analytes, and/or changes in one or more environmental conditions. As noted above, the analyte monitoring device 104 may be configured to produce data describing a single analyte (e.g., glucose) or multiple analytes.

In one or more implementations, the sensor module 204 may include a processor and memory (not shown). The sensor module 204, by leveraging the processor, may generate analyte measurements 212 based on the communications with the analyte sensor 202 that are indicative of the above-discussed changes. Based on the above-noted communications from the analyte sensor 202, the sensor module 204 is further configured to generate communicable packages of data that include at least one analyte measurement 212. In this example 200, the analyte data 118 represents these packages of data. Additionally or alternatively, the sensor module 204 may configure the analyte data 118 to include additional data, including, by way of example, supplemental sensor information 214. The supplemental sensor information 214 may include a sensor identifier, a sensor status, temperatures that correspond to the analyte measurements 212, measurements of other analytes that correspond to the analyte measurements 212, and so forth. It is to be appreciated that supplemental sensor information 214 may include a variety of data that supplements at least one analyte measurement 212 without departing from the spirit or scope of the described techniques.

In implementations where the analyte monitoring device 104 is configured for wireless transmission, the transmitter 208 may transmit the analyte data 118 as a stream of data to a computing device. Alternatively or additionally, the sensor module 204 may buffer the analyte measurements 212 and/or the supplemental sensor information 214 (e.g., in memory of the sensor module 204 and/or other physical computer-readable storage media of the analyte monitoring device 104) and cause the transmitter 208 to transmit the buffered analyte data 118 later at various regular or irregular intervals, e.g., time intervals (approximately every second, approximately every thirty seconds, approximately every minute, approximately every five minutes, approximately every hour, and so on), storage intervals (when the buffered analyte measurements 212 and/or supplemental sensor information 214 reach a threshold amount of data or a number of measurements), and so forth. It should be appreciated that in implementations the analyte monitoring device 104 can vary in numerous ways from the example described above without departing from the spirit or scope of the described techniques.

Having discussed one example of an analyte monitoring device, consider now the following discussion of one example medicament delivery system.

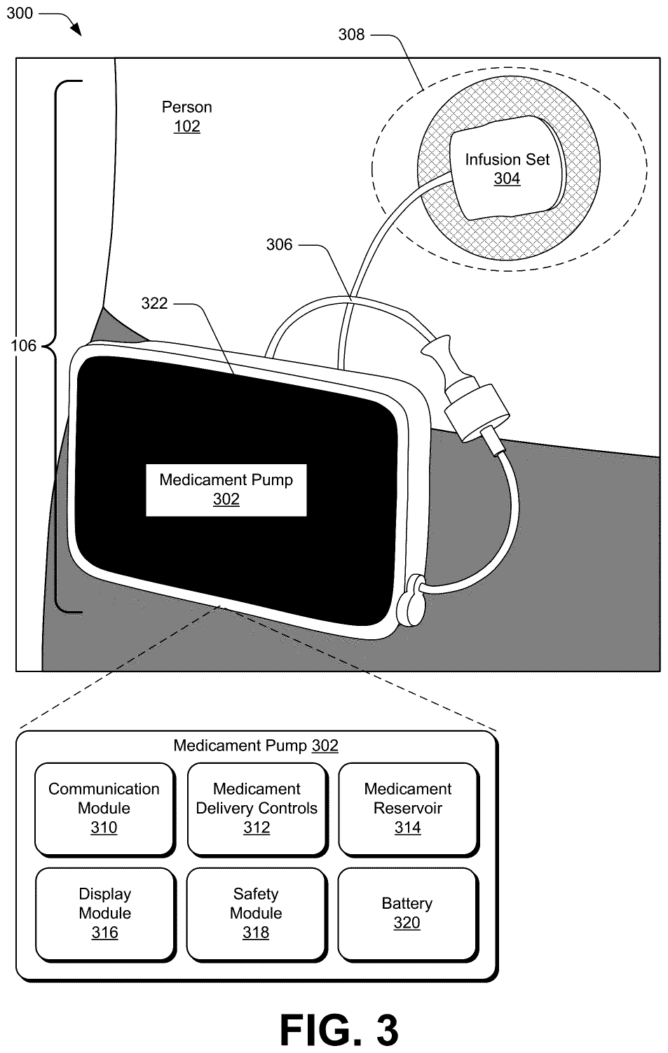

FIG. 3 depicts an example implementation 300 of the medicament delivery system 106 in greater detail.

In the example implementation 300, the medicament delivery system 106 includes a medicament pump 302 and infusion set 304. Although the infusion set 304 is depicted with tubing 306 connected to the medicament pump 302, in one or more implementations the infusion set 304 may be tubeless. Although depicted as pump in this example, the medicament delivery system 106 may be configured in various ways in accordance with the described techniques, examples of which include a pen or a set of pens, an inhaler, a patch, one or more syringes, and a mouth piece.

Broadly, the infusion set 304 is an apparatus configured to subcutaneously deliver medicament—pumped to the infusion set 304 by the medicament pump 302—into the person 102 for absorption by the person 102's bloodstream. In this way, the delivered medicament may be used by the person 102's body to maintain balanced analyte levels, e.g., within a target range of analyte measurements. In one or more implementations, the infusion set 304 includes a cannula that is inserted subcutaneously into skin, such as at infusion site 308 of the person 102 in the example implementation 300. Accordingly, the infusion set 304 may administer doses of medicament through the skin of the person 102, e.g., in a continuous manner and at programmable rates. As discussed herein, for instance, basal and/or bolus doses of insulin may be administered through the skin of the person 102 via the infusion set 304. Alternatively or in addition, the medicament delivery system 106 may administer medicament to the person 102 based on user input, e.g., input received via a user interface of one or more doses of medicament to deliver and/or input received via a computing device 108.

As illustrated, the infusion set 304 includes an adhesive pad which affixes the apparatus to the person 102 for a period of time. In one or more implementations, the infusion set 304 is applied to the infusion site 308 using a separate applicator (not shown). In operation, this applicator may inject the infusion set 304's cannula into the person 102's skin at the infusion site 308 and also affix the adhesive pad to the infusion site 308 to secure the infusion set 304 to the person 102 for a period of use. In at least some implementations, infusion sets may be disposable such that they are designed for removal after a prescribed and/or recommended period of time and for replacement with a new set that is applied to the person 102 and attached to the medicament pump 302. In any case, the medicament pump 302 is configured to deliver medicament doses to the person 102 via infusion sets, such as the illustrated infusion set 304.

In the example implementation 300, the medicament pump 302 includes communication module 310, medicament delivery controls 312, medicament reservoir 314, display module 316, safety module 318, and battery 320. In implementation, the medicament pump 302 may be configured in various ways, such as with some of these components while others are housed or otherwise implemented in separate devices. Alternately or additionally, the medicament pump 302 may include additional or alternate components without departing from the spirit or scope of the techniques described herein.

The communication module 310 is configured to transmit data to and receive data from other devices, such as the computing device 108 and/or the delivery device control system 110 (which may be at least partially included at the computing device 108 in one or more variations). The communication module 310 establishes communicative couplings with such other devices to enable transmission and receipt of data. By way of example, the communication module 310 may establish, or otherwise facilitate establishing, links or channels of communication with those other devices. The links or channels may be configured in various ways, including but not limited to Bluetooth (e.g., Bluetooth Low Energy links), Near Field Communication (NFC), 5G or other cellular, and Wi-Fi, to name just a few. Such communicative couplings enable the medicament pump 302 to communicate over different networks, such as the network 116 and/or securely within a closed loop system which includes, for example, the analyte monitoring device 104 and at least one computing device 108.

Once a communicative coupling is established, the communication module 310 can cause data to be transmitted over the established coupling and/or can receive data from other devices over the established coupling. Additionally or alternately, the communication module 310 may be configured to establish connections over wired communication channels—such as via a USB cord connected to the medicament pump 302 and another device—and also configured to transmit and/or receive data over such a wired coupling. The communication module 310 may be configured in various ways to enable the medicament pump 302 to communicate with other devices.

As one example, the communication module 310 enables the medicament pump 302 to receive the instructions 122 and/or other instructions from the computing device 108 and/or the delivery device control system 110 for controlling delivery of medicament to the person 102. For instance, the communication module 310 enables the medicament pump 302 to receive instructions that instruct the medicament pump 302 to suspend delivery of medicament to the person 102, subsequently resume delivery of medicament to the person, deliver an amount of medicament to the person 102 that is determined based, in part, on an amount of time that delivery of the medicament is suspended, and so forth. Alternatively or in addition, the communication module 310 enables the medicament pump 302 to receive instructions regarding delivery of a basal rate of insulin to the person 102, updates to the basal rate of insulin, delivery of bolus doses of insulin to the person 102 (e.g., an amount to bolus within a limited time), and so forth. The computing device 108 may send a variety of communications to the medicament delivery system 106 for controlling medicament delivery without departing from the spirit or scope of the techniques described herein.

The medicament delivery controls 312 represent any hardware, software, and/or mechanical components of the medicament pump 302 that cause medicament to be pumped (or otherwise extracted) from the medicament reservoir 314 so that it flows through the infusion set 304 and into the person 102. Moreover, the medicament delivery controls 312 are further configured to cause the medicament to be pumped or otherwise extracted from the medicament reservoir 314 in accordance with medicament dose instructions, e.g., the instructions 122 from the delivery device control system 110 which specify to suspend and/or resume one or more deliveries of the medicament. The medicament reservoir 314 is configured to house an amount of medicament, which by leveraging the functionality of the medicament pump 302 may be subcutaneously delivered via the infusion set 304. The medicament reservoir 314 may be replaceable or otherwise configured so that the amount of medicament can be replenished in the medicament reservoir 314. The medicament reservoir 314 may be configured in various ways (e.g., different shapes, different materials, differently removable, and so on) without departing from the spirit or scope of the described techniques.

The display module 316 is configured to cause display of information via a display device 322 of the medicament pump 302. The display module 316 may generate one or more user interfaces for display via the display device 322. By way of example, the display module 316 may cause display via the display device of a user interface for setting up a wireless connection with the computing device 108. Additionally or alternately, the display module 316 may cause the display device 322 to display analyte measurements (e.g., in a similar manner as they are displayed via a health monitoring application of the computing device 108), trend arrows (e.g., regarding an identified trend in the analyte measurements), alerts (e.g., that medicament delivery is suspended, that medicament delivery is resuming or resumed, about the analyte measurements, other physiological conditions, operability of the medicament delivery system 106, operability of components of the delivery device control system 110, status of a wireless connection with the computing device 108, and so on), pump set up interfaces, indications of being out of communication range from different devices (e.g., the computing device 108 or the analyte monitoring device 104), and so forth. The display module 316 may thus cause a variety of information to be displayed via the display device 322 of the medicament pump 302.

Safety module 318 is configured to provide one or more safeguards to control delivery of medicament so that the delivery is not harmful; in other words, so that the medicament is delivered safely. By way of example, the safety module 318 may contain or otherwise enforce delivery limits, such as maximum and minimum rates of delivery over different periods of time. These limits are effective to prevent erroneous delivery instructions from the delivery device control system 110 from harming the person 102, such as when an error transmitting the instructions affects their contents or when an error in a prediction made by one or more machine learning models dangerously affects those instructions. For instance, the safety module 318 may limit an amount or rate of medicament that the medicament pump 302 delivers to a threshold amount or threshold rate, even if instructions received from the delivery device control system 110 instruct the medicament pump 302 to deliver more than the threshold amount. Similarly, the safety module 318 may also prevent the medicament pump 302 from delivering less than a threshold amount of medicament or delivering medicament at less than a threshold rate, even if instructions received from the computing device 108 instruct the medicament pump 302 to deliver less than the threshold amount.

In addition, the safety module 318 is configured to continue operating the medicament pump 302 in the absence of instructions from the delivery device control system 110 or the computing device 108, e.g., in the absence of instructions describing how much medicament to deliver and when. The safety module 318 may be configured to continue operating the medicament pump 302 to deliver medicament to the person 102 when the delivery device control system 110 is out of communication range, for example. The safety module 318 may have access to logic, default settings, or settings entered as part of a set up process, to name just a few, that control how much medicament the medicament pump 302 is to deliver when instructions are not being received from the delivery device control system 110. The safety module 318 may perform various additional or different safeguards to ensure that an amount of medicament delivered is not harmful to the person 102.

The battery 320 is configured to provide power for operating the medicament pump 302, such as to power the communication module 310 to send and receive data, to power the medicament delivery controls 312 to cause delivery of medicament from the medicament reservoir 314 to the person 102 via the infusion set 304, to power the display module 316 to display information via the display device 322, and so forth. The battery 320 may be rechargeable (e.g., via a USB charging port or wirelessly) or replaceable. It is to be appreciated that the battery 320 may be configured in various ways.

Although not depicted, the medicament delivery system 106 or another device (e.g., the analyte monitoring device 104) may be configured with a medicament sensor (e.g., an insulin sensor). Such a medicament sensor may be applied to skin or inserted subcutaneously to measure a systemic level of the medicament, e.g., in the person 102. Accordingly, the medicament sensor may be included as part of the infusion set 304, the analyte monitoring device 104, or may be separately applied. In any case, such a sensor may be used in connection with the safety module 318 and/or with dose prediction functionality. In this way, medicament measurements produced using the medicament sensor can be used to prevent or detect an overdose in the medicament, e.g., to prevent a person who is receiving insulin from experiencing an episode of hypoglycemia. By way of example, the safety module 318 may shut off medicament delivery by the medicament pump 302 based on the medicament measurements, e.g., if the safety module 318 detects that the level of medicament surpasses a predetermined threshold. Based on the medicament measurements, the delivery device control system 110 may also or alternately send instructions to the medicament delivery system 106, instructing it to cease or temporarily suspend medicament delivery. In one or more implementations, the safety module 318 and/or the computing device 108 may also trigger alerts if the level of medicament surpasses the predetermined threshold. Physiologically, different thresholds may be determined for different persons based on their medicament sensitivities (e.g., insulin sensitivities), such that the above-discussed shut down, alerts, or cease to medicament delivery may be triggered at different medicament levels for the different persons.

Having considered an example of an environment and example devices, consider now a discussion of some examples of details of the techniques for automatic suspension and resumption of medicament delivery in accordance with one or more implementations.

Automatic Suspension and Resumption of Medicament Delivery

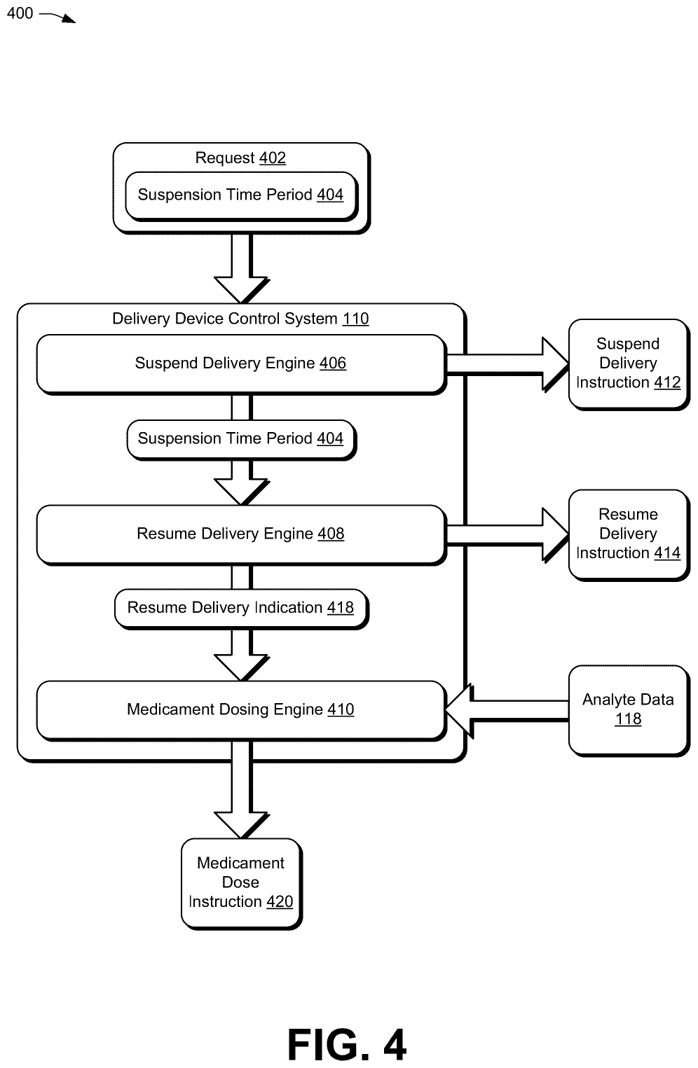

FIG. 4 depicts an example 400 of a system that suspends and resumes delivery of a medicament based on a request.

In the illustrated example 400, the system includes the delivery device control system 110. The delivery device control system 110 is depicted obtaining a request 402 to suspend delivery of a medicament, e.g., to suspend delivery of a medicament via the medicament delivery system 106. The request 402 includes suspension time period 404 in this example, however, in variations the request may not include the suspension time period 404. Instead, the delivery device control system 110 may determine the suspension time period 404 in one or more variations.

In one or more implementations, the request 402 is responsive to or otherwise based on receiving user input. For example, user input may be received via a user interface of the medicament delivery system 106 or the computing device 108 in relation to one or more controls (e.g., buttons, menus, fields, and so forth), which are operable to request that delivery of the medicament be suspended. In variations, user input may be received to specify the suspension time period 404, such as in relation to those same controls or one or more additional controls. Responsive to receiving the request 402 (e.g., based on user input), the delivery device control system 110 causes delivery of the medicament to be temporarily suspended.

In this particular example, the delivery device control system 110 includes a suspend delivery engine 406, a resume delivery engine 408, and a medicament dosing engine 410. Although the delivery device control system 110 is depicted with these components, in variations, the delivery device control system 110 includes more, fewer, or different components without departing from the spirit or scope of the described techniques. Accordingly, the functionality described above and below may be performed by one or more different components than described herein and/or by the delivery device control system 110.

In accordance with the described techniques, the suspend delivery engine 406 determines a time when to suspend delivery of the medicament to the user. In this example, for instance, the suspend delivery engine 406 determines to suspend delivery of the medicament responsive to receiving the request 402, e.g., the suspend delivery engine 406 determines to suspend delivery of the medicament substantially in real-time after the request 402 is received or an amount of time after the request 402 is received. Alternatively or in addition, the suspend delivery engine 406 is configured to process the request 402, extract a scheduled time specified in the request 402 (not shown), and determine to suspend delivery at the specified time.

The suspend delivery engine 406 is also configured to output a suspend delivery instruction 412. In one or more implementations, the suspend delivery engine 406 or the delivery device control system 110 communicates the suspend delivery instruction 412 to the medicament delivery system 106. Broadly, the suspend delivery instruction 412 instructs the medicament delivery system 106 to suspend delivery of a medicament at the time determined by the suspend delivery engine 406. In one or more implementations, the suspend delivery instruction 412 causes the medicament delivery system 106 to suspend delivery of the medicament substantially immediately upon receiving the instruction. Alternatively or in addition, the suspend delivery instruction 412 includes a specified time to suspend delivery, such that the medicament delivery system 106 suspends delivery of the medicament at the specified time.

The resume delivery engine 408 is depicted receiving the suspension time period 404 as input. In one or more implementations, the resume delivery engine 408 determines a time when to resume delivery of the medicament to the user. In this example, for instance, the resume delivery engine 408 determines the time when to resume delivery of the medicament to the user based on the suspension time period 404. In one or more variations, the resume delivery engine 408 computes the time to resume delivery of the medicament based on a time when the delivery of medicament is suspended and based on an amount of time of the suspension time period 404, e.g., the resume delivery engine 408 adds the suspension time period 404 to the time when the delivery of the medicament is suspended. Accordingly, in one or more implementations, in addition to receiving the suspension time period 404, the resume delivery engine 408 also receives (or otherwise has access to) the time when delivery of the medicament is suspended.

In accordance with the described techniques, the resume delivery engine 408 outputs a resume delivery instruction 414 based on the determined time to resume delivery of the medicament, which may further be based on the suspension time period 404 as discussed above. In one or more implementations, the resume delivery engine 408 or the delivery device control system 110 communicates the resume delivery instruction 414 to the medicament delivery system 106. Broadly, the resume delivery instruction 414 instructs the medicament delivery system 106 to resume delivery of the medicament at a time subsequent to the time when the medicament delivery is suspended. In one or more implementations, the resume delivery instruction 414 causes the medicament delivery system 106 to resume delivery of the medicament substantially upon receiving the instruction. Alternatively or in addition, the resume delivery instruction 414 includes a specified time to resume delivery, such that the medicament delivery system 106 resumes delivery of the medicament at the specified time, e.g., after delivery of the medicament has been suspended for the suspension time period 404.

In the illustrated example 400, the resume delivery engine 408 is also depicted outputting resume delivery indication 418. In one or more implementations, the resume delivery indication 418 may correspond to the resume delivery instruction 414 or it may be a different communication. Additionally, the medicament dosing engine 410 is depicted receiving the resume delivery indication 418. Broadly, the resume delivery indication 418 indicates to the medicament dosing engine 410 that the medicament delivery system 106 is resuming delivery of a medicament to a user (e.g., the person 102). Based on receipt of the resume delivery indication 418, the medicament delivery engine 410 determines a dose of the medicament to deliver to the user. The medicament dose instruction 420 corresponds to the determined dose and instructs the medicament delivery system 106 to deliver the determined dose after it resumes delivery of the medicament, e.g., according to the resume delivery instruction 414. In short, the resume delivery instruction 414 instructs the medicament delivery system 106 when to resume delivery of the medicament (e.g., a time to resume delivery), and the medicament dose instruction 420 instructs the medicament delivery system 106 how much medicament to deliver (e.g., an amount to deliver over time). Although not depicted in the subsequent illustrations, the medicament dosing engine 410 may be used in various implementations to instruct the medicament delivery system 106 regarding how much medicament to deliver before delivery of the medicament is suspended and/or how much medicament to deliver after delivery of the medicament is resumed.

In one or more implementations, the medicament dosing engine 410 determines the dosage to deliver upon resuming delivery based on various data, including one or more of the analyte data 118, the suspension time period 404, a time when the delivery is suspended, a subsequent time when the delivery is resumed, and the sensor data 120, to name just a few. The medicament dosing engine 410 may also determine doses to deliver based on “higher-level” determinations made from such data, such as one or more activities the user performed while delivery of the medicament was suspended. This is because activities the user performs during suspension of medicament delivery, such as exercise, can impact how much medicament should be delivered to the user so that suitable analyte levels are maintained. The medicament dosing engine 410 may also determine doses based on historical data about the user and/or historical data of at least one other user associated with the one or more activities performed during delivery suspension. The medicament dosing engine 410 may determine a dose for delivery based on a variety of data without departing from the spirit or scope of the described techniques.

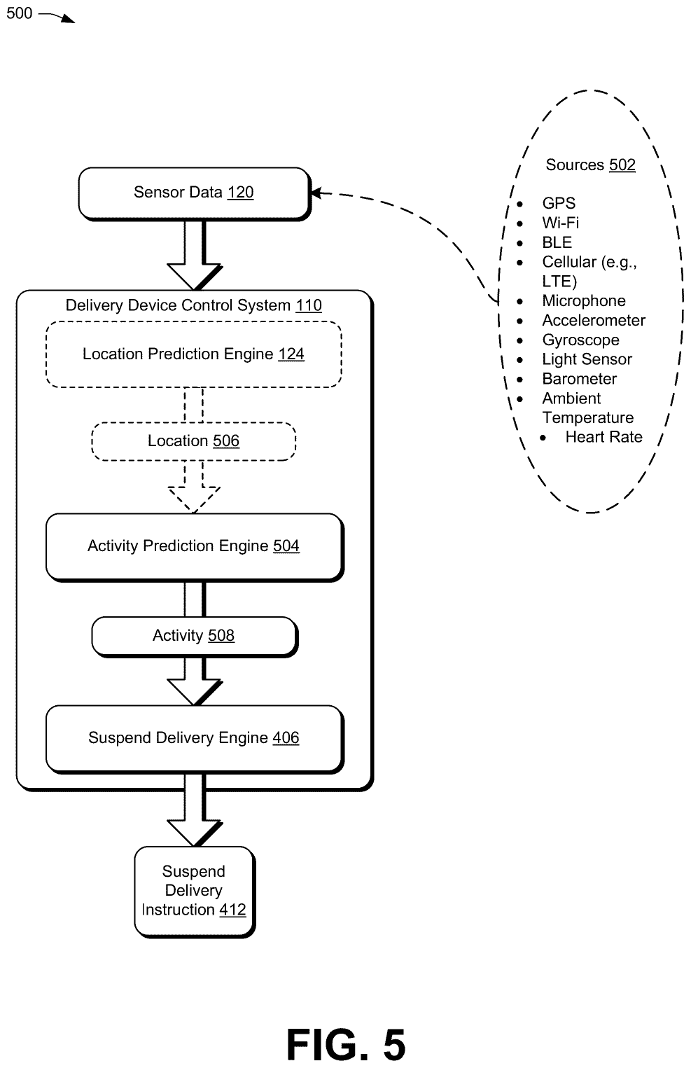

FIG. 5 depicts an example 500 of a system that suspends delivery of a medicament automatically based on sensor data. The illustrated example 500 includes the delivery device control system 110. In this example 500, the delivery device control system 110 suspends delivery of a medicament automatically based on the sensor data 120. For example, the delivery device control system 110 causes delivery of the medicament to be suspended based on the sensor data 120 and without receiving user input explicitly specifying to suspend medicament delivery. This contrasts with the previously discussed example 400 where the request 402 to suspend delivery is received.

In this example 500, the delivery device control system 110 is depicted obtaining the sensor data 120. In accordance with the described techniques, the delivery device control system 110 receives the sensor data 120 from various sources 502, examples of which include global positioning system (GPS) data, Wi-Fi information (e.g., service set identifiers (SSIDs)), Bluetooth Low Energy (BLE) information, data produced using a cellular antenna (e.g., long term evolution (LTE)), microphone data (e.g., sound data), accelerometer data, gyroscope data, magnetometer data, barometer data, ambient or internal temperature data (e.g., produced using temperature sensors), light data (e.g., captured using a device's camera), heart rate data (e.g., produced by a smartphone and/or a smart watch), proximity data (e.g., between devices), humidity data, analyte data (one or more different analytes or different data about a same analyte from different sources), and medicament data, to name just a few. Additional examples of the sensor data 120 from the various sources 502 includes, but is not limited to, measurements of various detected signals (e.g., biopotential measurements such as electrocardiogram (ECG), electromyography (EMG), or electroencephalogram (EEG); acceleration experienced by the person at a location where the analyte augmentation wearable is worn; and optical signals such as photoplethysmogram (PPG) that detect changes in blood volume), measurements of various physiological conditions (e.g., perspiration, temperature, heart rate, oxygen saturation (SpO₂)), or indications of detected events (e.g., exceeding or falling below a threshold, detecting the presence or absence of a particular compound), to name just a few. As noted above, the medicament delivery system 106, the computing device 108, and the IoT 114 may be the sources 502 of such data. Alternatively or in addition, the delivery device control system 110 receives the sensor data 120 from other sources 502 which may include or be associated with one or more various types of sensors.

In this example 500, the delivery device control system 110 includes the location prediction engine 124, an activity prediction engine 504, and the suspend delivery engine 406. As noted above and below, the delivery device control system 110 may include more, fewer, or different components in variations without departing from the spirit or scope of the described techniques, and the functionality discussed herein may be performed by one or more different components than described.

In one or more implementations, the delivery device control system 110 determines when to cause delivery of a medicament to be suspended based on predicting or otherwise detecting a location 506 of a user and predicting or otherwise detecting an activity 508 being performed by the user. In one or more implementations, the delivery device control system 110 determines when to cause delivery to be suspended based on the location 506. Alternatively or in addition, the delivery device control system 110 determines when to cause delivery to be suspended based on the activity 508. In one or more implementations, the activity 508 is determined based on the location 506, while in at least one other implementation, the activity is determined without using the location 506 of the user. In the illustrated example 500, the location prediction engine 124 and the location 506 are depicted with dashed lines to indicate that they are optional in at least one variation.

In the context of the illustrated example 500, the location prediction engine 124 determines or otherwise predicts the location 506 of the user (e.g., the person 102) based on the sensor data 120. The location prediction engine 124 outputs the location 506. The activity prediction engine 504 determines or otherwise predicts the activity 508 of the user, such as the activity the user is performing at a current time. Examples of activities include, but are not limited to, eating, sleeping, exercising (e.g., aerobic, anerobic, partially aerobic and partially anerobic, high-intensity interval training, running, rowing, hiking, biking, weightlifting, yoga, Pilates, sports, and so forth), working (e.g., potentially causing stress), bathing or showering, watching a movie, watching television, attending an event (e.g., a sporting event or a concert), dancing, reading, cleaning, taking care of children, or driving, to name just a few.

As mentioned above, the activity prediction engine 504 optionally determines the activity 508 of the user based on the location 506 of the user. By way of example, the presence of a user at the gym, within a shower, in bed, and/or sitting in a chair in a workspace, may be informative with respect to the activity 508 being performed. Alternatively or in addition, the activity prediction engine 504 determines the activity 508 of the user based on the sensor data 120, such as based on heart rate data (indicating exercise).

Based on the activity 508, the suspend delivery engine 406 determines to suspend delivery of medicament to the user. In one or more variations, this includes a time at which to suspend the delivery. Further, the suspend delivery engine 406 outputs the suspend delivery instruction 412 to cause delivery to be suspended, e.g., at the time. For example, the suspend delivery engine 406 outputs the suspend delivery instruction 412 to the medicament delivery system 106 and the instruction causes the medicament delivery system 106 to suspend delivery of the medicament to the user. The suspend delivery instruction 412 is one example of the instructions 122.

In terms of how the location prediction engine 124 predicts the location 506 of a user, in one or more implementations, the location prediction engine 124 predicts the location 506 based on at least two types of sensor data, e.g., first sensor data and the second sensor data. For instance, the location prediction engine 124 predicts the location 506 based on both GPS data and Wi-Fi information (e.g., an SSID of a network accessible to the computing device 108), based on both Wi-Fi information and sound data, based on both Wi-Fi information and light data, based on both Wi-Fi information and heart rate data, to name just a few. It is to be appreciated that the location prediction engine 124 can be configured to receive as input, and process, various combinations of at least two types of the data described above and below to predict the location 506 of a user. Certainly, the location prediction engine 124 can use more than two types of data to predict the location 506 of the user in one or more implementations, e.g., more than first sensor data and second sensor data.

In at least one implementation, the location prediction engine 124 predicts the location 506 of a user (e.g., the person 102) based on a location of a computing device 108 associated with the user. For example, the location prediction engine 124 predicts the location 506 of the user based on the location of a mobile phone or a smart watch of the user. In such implementations, this is based on the assumption that the user is proximate the associated computing device, e.g., the computing device is worn by the user or carried in the user's clothing or an accessory. In other words, the location prediction engine 124 predicts (or detects) the location of the user's computing device 108 (e.g., the user's mobile phone or smart watch) then ascribes the device's location to the user. Alternatively or in addition, the location prediction engine 124 predicts (or detects) the location of the user's computing device 108 (e.g., the user's mobile phone or smart watch) then uses sensor data 120 (e.g., proximity data, infrared data, temperature data, etc.) obtained from the computing device 108 (or other sources) to further refine the location and/or determine a location of the user relative to the computing device 108. This may be the case when the computing device 108 is not worn by the user or is not “on” the user, such as when the user is sleeping or showering. In one or more implementations, the location prediction engine 124 generates a “coarse” prediction of the location 506 using first sensor data and generates a “refined” prediction of the location 506 using second sensor data (and based on the coarse location prediction).

The location prediction engine 124 may be configured in a variety of ways without departing from the spirit or scope of the described techniques. For example, the location prediction engine 124 may be configured as or include one or more machine learning models. Alternatively or in addition, the location prediction engine 124 includes or has access to a mapping of locations (e.g., restaurants, gyms, houses, stores, roads, and other businesses) to geographic coordinates, such that given precise geographic coordinates the location prediction engine 124 can ascribe the user's presence to a location according to the mapping.

In at least one implementation, a first portion of the sensor data 120 may not be suitable to enable the location prediction engine 124 to predict the user's geographic coordinates precisely enough to discriminate between at least two proximate locations. Instead, the first portion of the sensor data 120 may be suitable to predict an area (e.g., a radius around a point) where the user is likely to be located, such that the area includes at least two candidate locations. Thus, in one or more implementations, the location prediction engine 124 uses at least a second portion of the sensor data 120 to discriminate between the candidate locations and select the location 506 from the multiple candidate locations. In a scenario where a bedroom and a bathroom neighbor one another, for instance, the location prediction engine 124 uses the second portion of sensor data 120 to narrow down the candidate locations to select either the bedroom or the bathroom. This is notable because the activities that users participate in at different locations can be associated with suspending delivery of a medicament (e.g., bathing in a bathroom) or delivering the medicament (e.g., sleeping in a bedroom). Similarly, consider an example where a restaurant and a gym are both located in a strip mall. In this scenario, it is important to be able to determine whether the user is at the gym (and exercising) or at the restaurant (and eating) because insulin may need to be suppressed if the user is exercising but increased if the user is eating.

In terms of how the activity prediction engine 504 predicts the activity 508 the user is participating in or likely to be participating in, in one or more implementations the activity prediction engine 504 determines and outputs the activity 508 based on the location 506. In one or more variations, the activity prediction engine 504 predicts the activity 508 based on the predicted location 506 without using other sensor data 120 (than is used to determine the location 506) or user interaction to confirm the activity 508. In at least one other variation, the activity prediction engine 504 predicts the activity 508 based on the predicted location 506 and at least one other type of data, e.g., the sensor data 120 and/or based on data describing user interaction with a device. Examples of such sensor data 120 include sound data (e.g., indicative of sounds confirming that a user is sleeping), light data (e.g., indicative of a dark location which is associated with a higher likelihood that the user is sleeping), accelerometer data (e.g., indicative of a positioning of a device on a nightstand which is associated with a higher likelihood that a user is sleeping), and so on. Examples of such user interaction include user interaction to confirm a predicted activity 508, to input the activity, and so forth. As mentioned above, in one or more implementations, the location prediction engine 124 predicts the activity 508 based on the sensor data 120 without receiving a predicted or otherwise determined location 506.

It is to be appreciated, that in some configurations the location prediction engine 124 detects the location 506 of the user in real time (or substantially real time) as the user's location changes and/or the activity prediction engine 504 predicts an activity 508 being performed by the user in real time (or substantially real time). Notably, not all activities are associated with suspending medicament delivery. Thus, in one or more implementations, the suspend delivery engine 406 only generates the suspend delivery instruction 412 in instances where the activity 508 predicted is associated with suspending medicament delivery (and delivery of the medicament has not yet been suspended).

In one or more implementations, the suspend delivery engine 406 is configured as a machine learning model that receives as input the activity 508 (and an activity state in some implementations) and outputs the suspend delivery instruction 412. In such implementations, the suspend delivery engine 406 may be trained using training data that pairs activities (and activity state in some implementations) with a tag indicating to deliver medicament while the activity is being performed or a tag indicating not to deliver medicament while the activity is being performed. By way of example, the machine learning model may be exposed to an activity (e.g., receive it as input) and attempt to output an indication of whether to deliver medicament during the activity or not. The system may then compare the output to the tag paired with the input in the training data. Based on this comparison, internal weights of the machine learning model may be adjusted. For instance, if the output matches the tag paired in the training data, then weights may be adjusted (e.g., strengthened) to encourage the output during future iterations. However, if the output does not match the tag paired in the training data, then the weights may be adjusted to discourage the output during future iterations. The machine learning model may be thusly trained over numerous iterations.

Alternatively or in addition, the suspend delivery engine 406 may obtain an indication of whether the activity 508 corresponds to an activity during which to deliver the medicament or an activity during which medicament is not to be delivered, such as from a mapping of activities (and states in some implementations) to delivery or non-delivery of the medicament. By way of example, a library may include a mapping that maps different activities to delivery or non-delivery of the medicament. This information may be stored in a database, for example. In one or more implementations, the mapping may be approved by one or more clinicians.

This information may be input to the system (e.g., into a database) and maintained. In one or more implementations, such a mapping may be maintained in computer-readable storage media of a computing device 108 of the user (e.g., the user's mobile phone). Alternatively or in addition, the mapping may be maintained remote from the computing device 108, such as in a database associated with the delivery device control system 110 and/or the health monitoring platform 112. Either or both of remote and local mappings may be updated as new activities are added. In one or more implementations, the mapping may be personalized to a user, e.g., to map activities to historical actions of the user to suspend or resume medicament delivery. In such implementations, the mapping may be provided by the user (e.g., via user input to the user's computing device), a health care provider (e.g., via a health care provider portal), or determined by the system based on the sensor data.

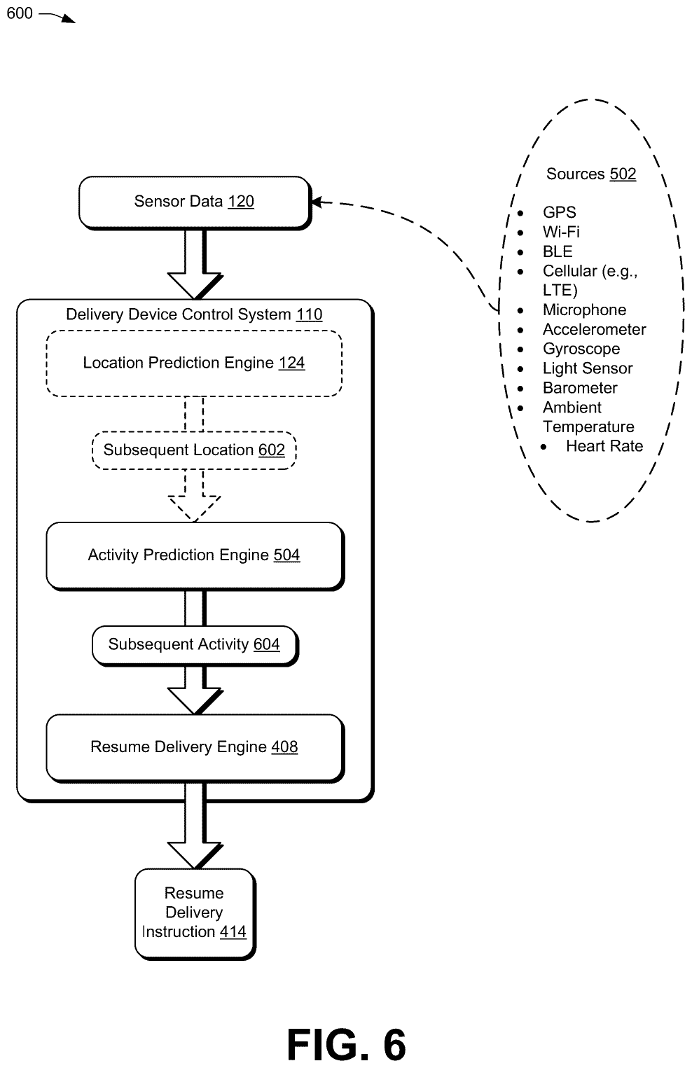

FIG. 6 depicts an example 600 of a system that resumes delivery of a medicament automatically based on sensor data. The illustrated example 600 includes the delivery device control system 110.

In this example 600, the delivery device control system 110 resumes delivery of a medicament automatically based on the sensor data 120. For example, the delivery device control system 110 causes delivery of the medicament to be resumed based on the sensor data 120 and without receiving user input explicitly to resume medicament delivery. This contrasts with the previously discussed example 400 where the request 402 to suspend the delivery includes a suspension time period 404, such that a time to resume delivery is determinable based on the suspension time period 404.

In this example 600, the delivery device control system 110 is depicted obtaining the sensor data 120, which as noted above, the delivery device control system 110 may obtain from a variety of sources 502. Further, the delivery device control system 110 includes the location prediction engine 124, the activity prediction engine 504, and the resume delivery engine 408. As noted above and below, the delivery device control system 110 may include more, fewer, or different components in variations without departing from the spirit or scope of the described techniques, and the functionality discussed herein may be performed by one or more different components than described.

In one or more implementations, the delivery device control system 110 determines when to cause delivery of a medicament to be resumed based on predicting or otherwise detecting a subsequent location 602 of a user and predicting or otherwise detecting a subsequent activity 604 being performed by the user. In one or more implementations, the subsequent location 602 is different from the location 506, based on which medicament delivery was automatically suspended. In one example, for instance, the location 506 corresponds to a shower and the subsequent location 602 corresponds to a different location from the shower, e.g., not the shower, a closet, or a bedroom. In another example, the location 506 corresponds to a gym and the subsequent location 602 corresponds to a different location from the gym, e.g., a parking lot of the gym, a locker room in a same facility as the gym, or on the road driving away from the gym.

Similarly, in one or more implementations, the subsequent activity 604 is different from the activity 508, based on which the medicament delivery was automatically suspended. In one example, for instance, the activity 508 corresponds to showering and the subsequent activity 604 corresponds to a different activity from showering, e.g., not showering, brushing hair, or dressing. In one example, for instance, the activity 508 corresponds to playing basketball and the subsequent activity 604 corresponds to a different activity from playing basketball, e.g., not playing basketball, walking to a locker room, walking to a parking lot, or driving a car.

In one or more implementations, the activity prediction engine 504 determines the subsequent activity 604 based at least in part on the subsequent location 602, while in at least one other implementation, the subsequent activity 604 is determined without using the subsequent location 602 of the user. In the illustrated example 600, the location prediction engine 124 and the subsequent location 602 are depicted with dashed lines to indicate they are optional in at least one variation.

In the context of the illustrated example 600, the location prediction engine 124 determines or otherwise predicts the subsequent location 602 of the user (e.g., the person 102) based on the sensor data 120. The location prediction engine 124 outputs the subsequent location 602. The activity prediction engine 504 determines or otherwise predicts the subsequent activity 604 of the user, such as the activity the user is performing at a current time. In one or more implementations, the subsequent activity 604 is an indication that the user is finished performing or is no longer performing the activity 508, e.g., a different “activity state” from a state of “during” the activity 508 (or “mid-activity”). Alternatively or in addition, the subsequent activity 604 corresponds to a different activity performed by the user.

Based on the subsequent activity 604, the resume delivery engine 408 determines to resume delivery of medicament to the user. Further the resume delivery engine 408 outputs the resume delivery instruction 414 to cause delivery of the medicament to be resumed automatically, e.g., without user interaction to resume the delivery. For example, the resume delivery engine 408 outputs the resume delivery instruction 414 to the medicament delivery system 106 and the instruction causes the medicament delivery system 106 to resume delivery of the medicament to the user (e.g., the person 102). The resume delivery instruction 414 is one example of the instructions 122.

In terms of how the location prediction engine 124 predicts the subsequent location 602, the location prediction engine 124 may do so in a similar manner as the location prediction engine 124 predicts the location 506. The activity prediction engine 504 may also predict the subsequent activity 604 in a similar manner as the activity prediction engine 504 predicts the activity 508. Notably, not all activities are associated with resuming medicament delivery. Thus, in one or more implementations, the resume delivery engine 408 only generates the resume delivery instruction 414 in instances where the subsequent activity 604 predicted is associated with resuming medicament delivery (and delivery of the medicament has not yet resumed).

In one or more implementations, the resume delivery engine 408 is configured as a machine learning model that receives as input the subsequent activity 604 (and an activity state in some implementations) and outputs the resume delivery instruction 414. In such implementations, the resume delivery engine 408 may be trained using training data that pairs activities (and activity state in some implementations) with a tag indicating to deliver medicament while the activity is being performed or a tag indicating not to deliver medicament while the activity is being performed.

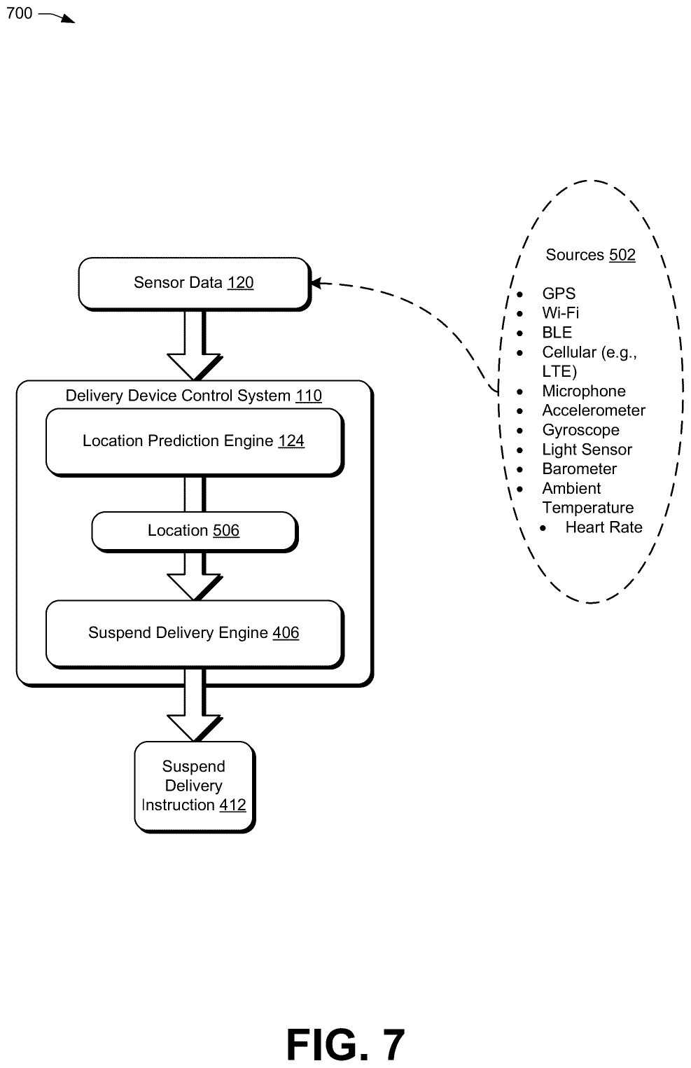

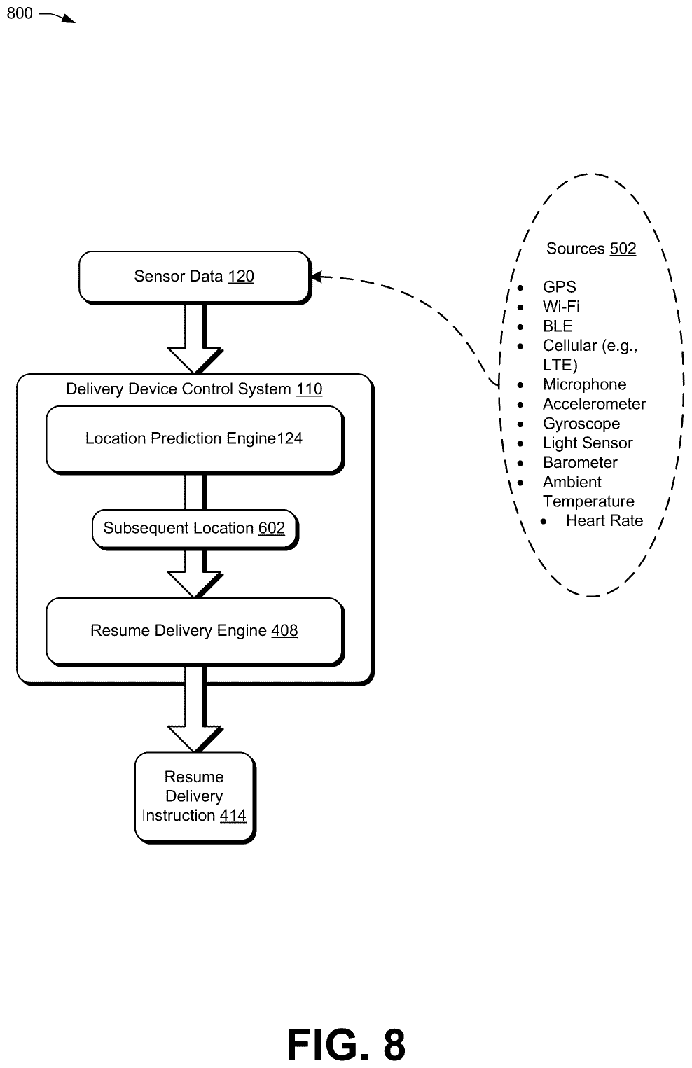

Alternatively or in addition, the resume delivery engine 408 may obtain an indication of whether the subsequent activity 604 corresponds to an activity during which to deliver the medicament or an activity during which medicament is not to be delivered, such as from a mapping of activities (and states in some implementations) to delivery or non-delivery of the medicament. By way of example, a library may include a mapping that maps different activities to delivery or non-delivery of the medicament. This information may be stored in a database, for example. In one or more implementations, the mapping may be approved by one or more clinicians. In the context of using determined locations to suspend and resume delivery of a medicament (e.g., without determining an activity), consider the following discussion of FIGS. 7 and 8.

FIG. 7 depicts an example 700 of a system that suspends delivery of a medicament automatically based on a location of a user determined using sensor data.

The illustrated example 700 includes the delivery device control system 110. In this example 700, the delivery device control system 110 suspends delivery of a medicament automatically based on the location 506, as determined based on the sensor data 120. In contrast to the example 500, the suspend delivery engine 406 is depicted receiving the location 506 rather than an activity. This represents that in one or more implementations, the delivery device control system 110 is configured to determine when to suspend delivery of a medicament based solely on the location 506, e.g., without using the activity 508. As discussed above and below, the location prediction engine 124 predicts the location 506 of the user based on the sensor data 120, which may be obtained from any of a variety of sources 502.

Based on the location 506, the suspend delivery engine 406 determines to suspend delivery of medicament to the user. In one or more variations, this includes a time at which to suspend the delivery. Further, the suspend delivery engine 406 outputs the suspend delivery instruction 412 to cause delivery to be suspended automatically, e.g., at the time. For example, the suspend delivery engine 406 outputs the suspend delivery instruction 412 to the medicament delivery system 106 and the instruction causes the medicament delivery system 106 to suspend delivery of the medicament to the user. As noted above, the suspend delivery instruction 412 is one example of the instructions 122.

FIG. 8 depicts an example 800 of a system that resumes delivery of a medicament automatically based on a subsequent location of a user determined using sensor data.

The illustrated example 800 includes the delivery device control system 110. In this example 800, the delivery device control system 110 resumes delivery of a medicament automatically based on the subsequent location 602, as determined based on the sensor data 120. In contrast to the example 600, the resume delivery engine 408 is depicted receiving the subsequent location 602 rather than a subsequent activity. This represents that in one or more implementations, the delivery device control system 110 is configured to determine when to resume delivery of a medicament based solely on the subsequent location 602 of the user, e.g., without using the subsequent activity 604. As discussed above and below, the location prediction engine 124 predicts the subsequent location 602 of the user based on the sensor data 120, which may be obtained from any of a variety of sources 502.

Based on the subsequent location 602, the resume delivery engine 408 determines to resume delivery of medicament to the user. In one or more variations, this includes a time at which to resume the delivery. Further, the resume delivery engine 408 outputs the resume delivery instruction 414 to cause delivery to be resumed automatically, e.g., at the time. For example, the resume delivery engine 408 outputs the resume delivery instruction 414 to the medicament delivery system 106 and the instruction causes the medicament delivery system 106 to resume delivery of the medicament to the user. As noted above, the resume delivery instruction 414 is one example of the instructions 122. Examples of user interfaces that can be output in connection with automatically suspending and resuming delivery of a medicament are discussed just below.

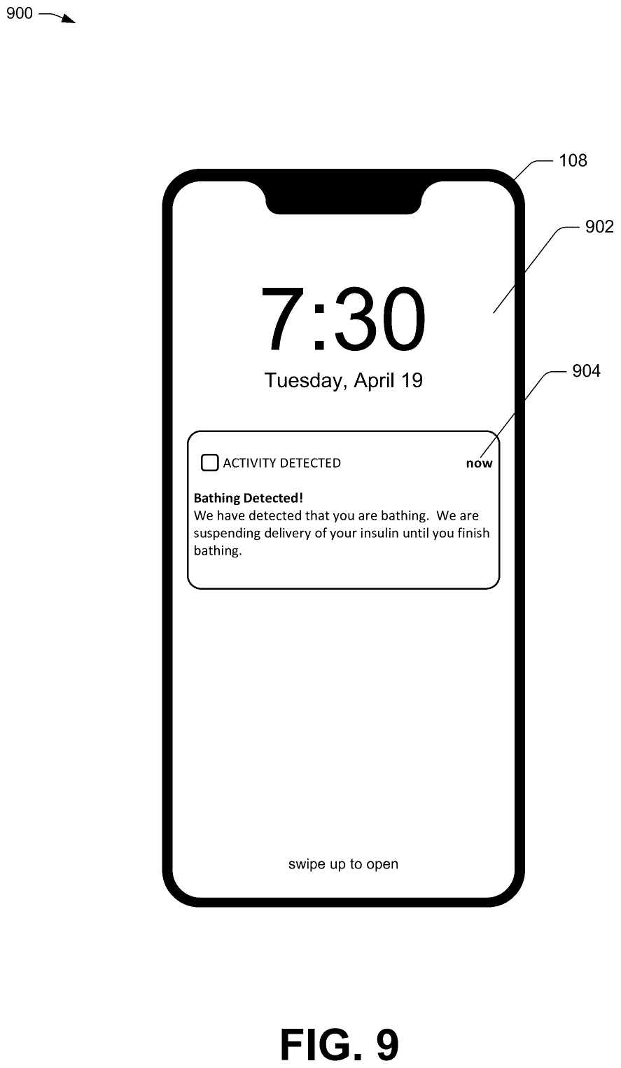

FIG. 9 depicts an example 900 of a user interface displaying a delivery suspension notification. The illustrated example 900 includes an example of the computing device 108 displaying an example user interface 902 via a display device, e.g., a touchscreen.

Here, the user interface 902 includes a delivery suspension notification 904 which indicates that delivery of a medicament (e.g., via the medicament delivery system 106) has been automatically suspended. The delivery suspension notification 904 informs a user about the suspension, since, due to the automatic suspension, the user may not know that delivery of the medicament has been suspended. Although the delivery suspension notification 904 is depicted being output (e.g., displayed) by the computing device 108, it is to be appreciated that the delivery suspension notification 904 may be output by any one or more of the analyte monitoring device 104, the medicament delivery system 106, or the computing device 108. The delivery suspension notification 904 may also be output by one or more additional devices without departing from the spirit or scope of the described techniques. Further, although the delivery suspension notification 904 is illustrated in this example 900 as being displayed, the delivery suspension notification 904 may be output in other ways and/or combined with other outputs, such as audio outputs (e.g., via a speaker) and tactile outputs (e.g., vibrations), without departing from the spirit or scope of the described techniques.

In this example, the activity corresponds to predicted bathing based on sensor data 120, which indicates, for instance, that the user is located in a bathtub or shower (e.g., location 506), is exposed to water and/or soap, is using products and/or articles associated with bathing, and so forth. Additionally or alternatively, the sensor data 120 includes sound data, e.g., capturing one or more sounds of bathing or showering.

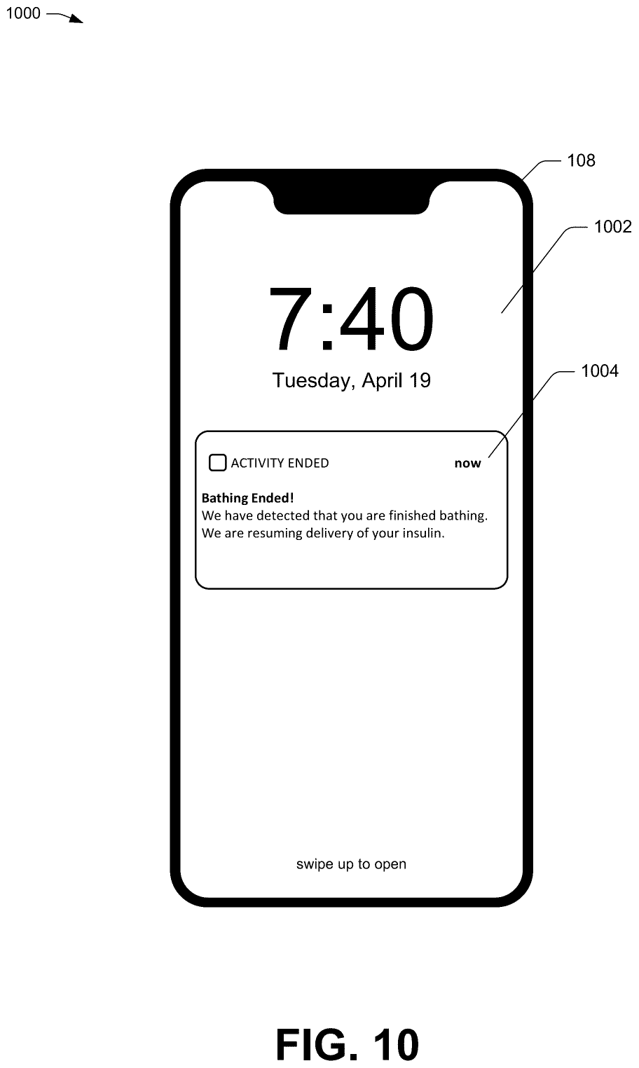

FIG. 10 depicts an example 1000 of a user interface displaying a delivery resumption notification. The illustrated example 1000 includes an example of the computing device 108 displaying an example user interface 1002 via a display device, e.g., a touchscreen.

Here, the user interface 1002 includes a delivery resumption notification 1004 which indicates that delivery of a medicament (e.g., via the medicament delivery system 106) has been automatically resumed. The delivery resumption notification 1004 informs a user about the resumed delivery, since, due to the automatic resumption, the user may not know that delivery of the medicament has been resumed. Although the delivery resumption notification 1004 is depicted being output (e.g., displayed) by the computing device 108, it is to be appreciated that the delivery resumption notification 1004 may be output by any one or more of the analyte monitoring device 104, the medicament delivery system 106, or the computing device 108. The delivery resumption notification 1004 may also be output by one or more additional devices without departing from the spirit or scope of the described techniques. Further, although the delivery resumption notification 1004 is illustrated in this example 1000 as being displayed, the delivery resumption notification 1004 may be output in other ways and/or combined with other outputs, such as audio outputs (e.g., via a speaker) and tactile outputs (e.g., vibrations), without departing from the spirit or scope of the described techniques.

In this example, the subsequent activity corresponds to a prediction that the user is no longer bathing based on sensor data 120, which indicates, for instance, that the user is not located in a bathtub or shower (e.g., subsequent location 602), is not exposed to water and/or soap, is not using products and/or articles associated with bathing, and so forth. Additionally or alternatively, the sensor data 120 includes sound data, e.g., capturing one or more sounds that correspond to post-bathing or post-showering activities or one or more sounds that indicate the user is no longer bathing or showering.

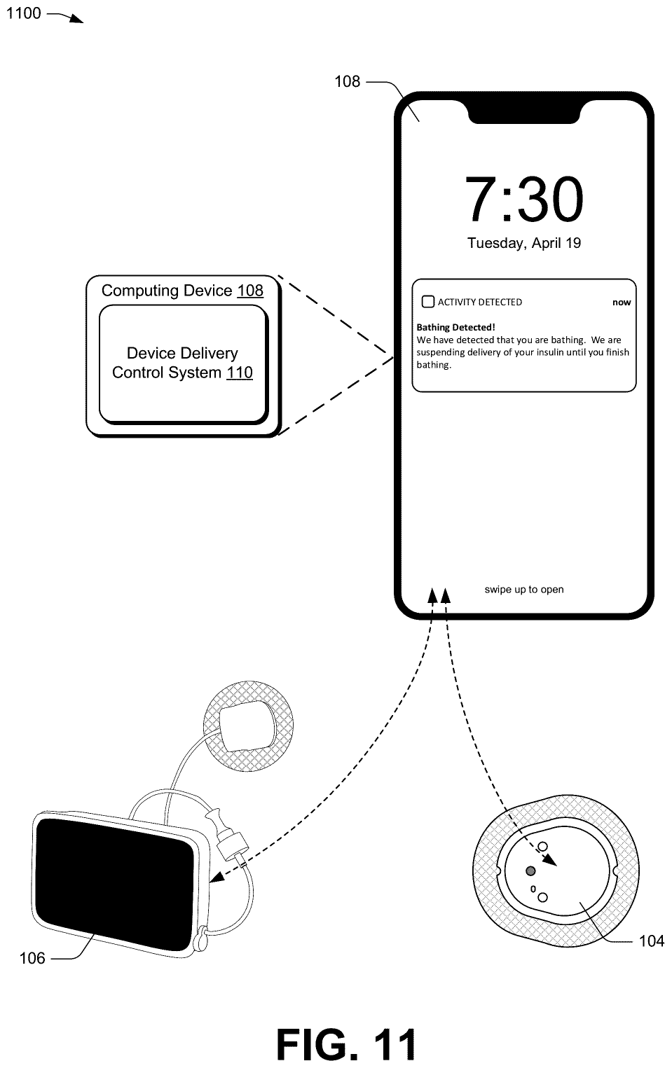

FIG. 11 depicts an example 1100 of a first combination of devices for implementing automatic suspension and resumption of medicament delivery. The illustrated example 1100 includes the analyte monitoring device 104, the medicament delivery system 106, and the computing device 108 having the delivery device control system 110.

In accordance with the described techniques, the analyte monitoring device 104, the medicament delivery system 106, and the computing device 108 may be communicably coupled, e.g., over the network 116 or via some other wireless connection (e.g., BLE), to implement any of the automatic suspension and resumption of medicament delivery techniques described above and below. In variations, the analyte monitoring device 104, the medicament delivery system 106, and the computing device 108 are operably connected to implement automatic suspension and resumption of medicament delivery as a closed loop system, an open loop system, or a partially open loop system. In this example, the medicament delivery system 106 is depicted as a wearable pump (e.g., insulin pump) having an infusion set that is applied to the person 102 and delivers medicament via the infusion set at an insertion site.

As a closed loop system, the analyte monitoring device 104, the medicament delivery system 106, and the computing device 108 are configured to monitor an analyte of the person 102 wearing the analyte monitoring device 104, and cause the medicament delivery system 106 to suspend delivery of a medicament automatically based on one or more of the location 506 of the person 102 and/or an activity that the person 102 is predicted to participate in without user interaction. Likewise, the analyte monitoring device 104, the medicament delivery system 106, and the computing device 108, when implemented as a closed-loop system, are configured to cause the medicament delivery system 106 to resume delivery of the medicament automatically based on one or more of the location 506 of the person 102 and/or an activity that the person 102 is predicted to participate in without user interaction.

By way of contrast, in a partially open system, a user may be required to validate suspension of medicament delivery before it is suspended by the system, e.g., the user may be required to provide approval of a recommended suspension of medicament delivery via a display of the analyte monitoring device 104, the medicament delivery system 106, and/or the computing device 108 before it is automatically suspended by the medicament delivery system 106. Once validated, the medicament delivery system 106 may suspend delivery of the medicament as recommended by the delivery device control system 110. In a partially open system, a user may also be required to validate resumption of medicament delivery before it is resumed by the system, e.g., the user may be required to provide approval of a recommended resumption of medicament delivery via a display of the analyte monitoring device 104, the medicament delivery system 106, and/or the computing device 108 before it is automatically resumed by the medicament delivery system 106.

In an open system, the delivery device control system 110 may simply cause one or more controls to be output (e.g., displayed via the analyte monitoring device 104, the medicament delivery system 106, or the computing device 108), which a user can interact with to generate a request 402 to suspend delivery of the medicament via the medicament delivery system 106.

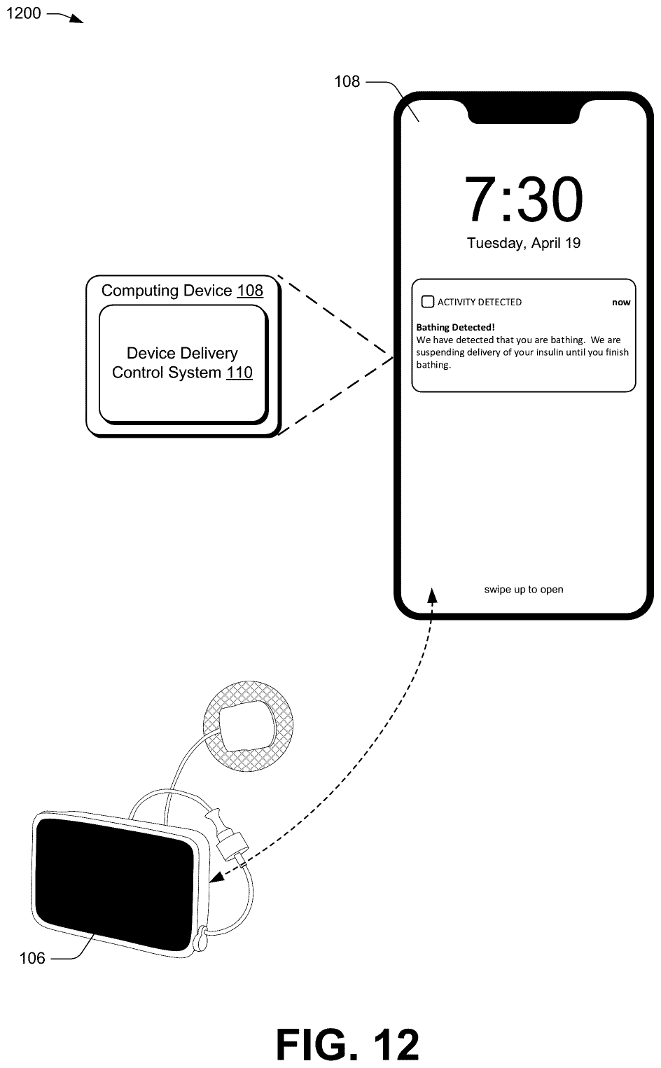

FIG. 12 depicts an example 1200 of a second combination of devices for implementing automatic suspension and resumption of medicament delivery. The illustrated example 1200 includes the medicament delivery system 106 and the computing device 108 having the delivery device control system 110.

The illustrated example 1200 does not include an analyte monitoring device 104, however. This represents a scenario where the medicament delivery system 106 and the computing device 108 are not operably connected to the analyte monitoring device 104. In such configurations, the delivery device control system 110 is capable of is capable of determining to suspend and resume delivery of a medicament automatically without using the analyte data 118. Instead, the delivery device control system 110 predicts a location 506 of a user associated with the computing device 108 and wearing the medicament delivery system 106 (e.g., a pump), and/or the delivery device control system 110 predicts an activity 508 of the user using other data, e.g., the sensor data 120. Examples of such other data are discussed above, but may include, for instance, GPS data and data produced by other sensors of the computing device. Based on the location and/or the activity predictions generated by the delivery device control system 110 using this other data, the delivery device control system 110 can further control the medicament delivery system 106 to suspend and resume delivery of a medicament automatically.

Like the example illustrated in FIG. 11, the medicament delivery system 106 and the computing device 108 may be operably connected to implement automatic suspension and resumption of medicament delivery as a closed loop system, an open loop system, or a partially open loop system.

Having discussed exemplary details of the techniques for automatic suspension and resumption of medicament delivery, consider now some examples of procedures to illustrate additional aspects of the techniques.

### Example Procedures

This section describes examples of procedures for automatic suspension and resumption of medicament delivery. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations the procedures are performed by a glycemic control system, such as delivery device control system 110.

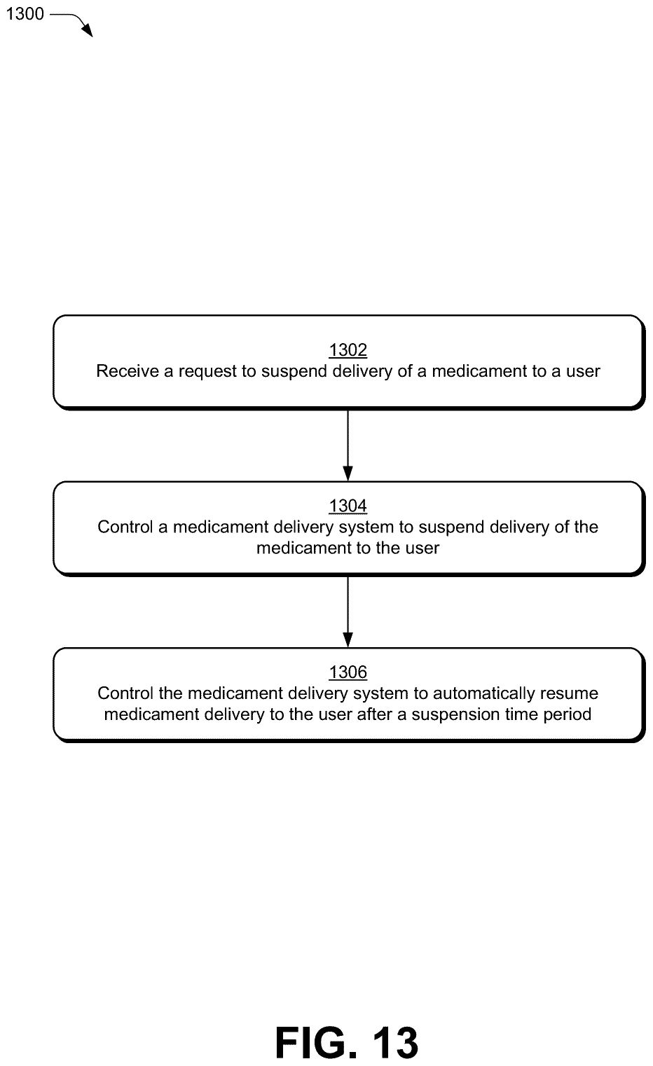

FIG. 13 depicts a procedure 1300 in an example implementation of automatic suspension and resumption of medicament delivery after a suspension time period.

A request to suspend delivery of a medicament to a user is received (block 1302). By way of example, the delivery device control system 110 receives a request 402 to suspend delivery of a medicament. In one or more implementations, the request 402 is responsive to or otherwise based on receiving user input. For example, user input may be received via a user interface of the medicament delivery system 106 or the computing device 108 in relation to one or more controls (e.g., buttons, menus, fields, and so forth), which are operable to request that delivery of the medicament be suspended

A medicament delivery system is controlled to suspend delivery of the medicament to the user (block 1304). By way of example, responsive to receiving the request 402 (e.g., based on user input), the delivery device control system 110 causes delivery of the medicament to be suspended. In one or more implementations, a suspend delivery engine 406 of the delivery device control system 110 determines a time at which delivery of the medicament is to be suspended, and outputs a suspend delivery instruction 412. The suspend delivery instruction is communicated to the medicament delivery system 106. Broadly, the suspend delivery instruction 412 instructs the medicament delivery system 106 to suspend delivery of a medicament at the time determined by the suspend delivery engine 406.

The medicament delivery system is controlled to automatically resume medicament delivery to the user after a suspension time period (block 1306). By way of example, the delivery device control system 110 controls the medicament delivery system 106 to automatically resume medicament delivery to the user after a suspension time period 404. In one or more implementations, the suspension time period 404 is specified by the user and received with the request 402. Alternatively, the suspension time period 404 may correspond to a predetermined period of time, e.g., 15 minutes, 30 minutes, 45 minutes, and so forth. In order to resume medicament delivery, a resume delivery engine 408 of the delivery device control system 110 outputs a resume delivery instruction 414 which is communicated to the medicament delivery system 106. Broadly, the resume delivery instruction 414 instructs the medicament delivery system 106 to resume delivery of the medicament at a time subsequent to the time when the medicament delivery is suspended.

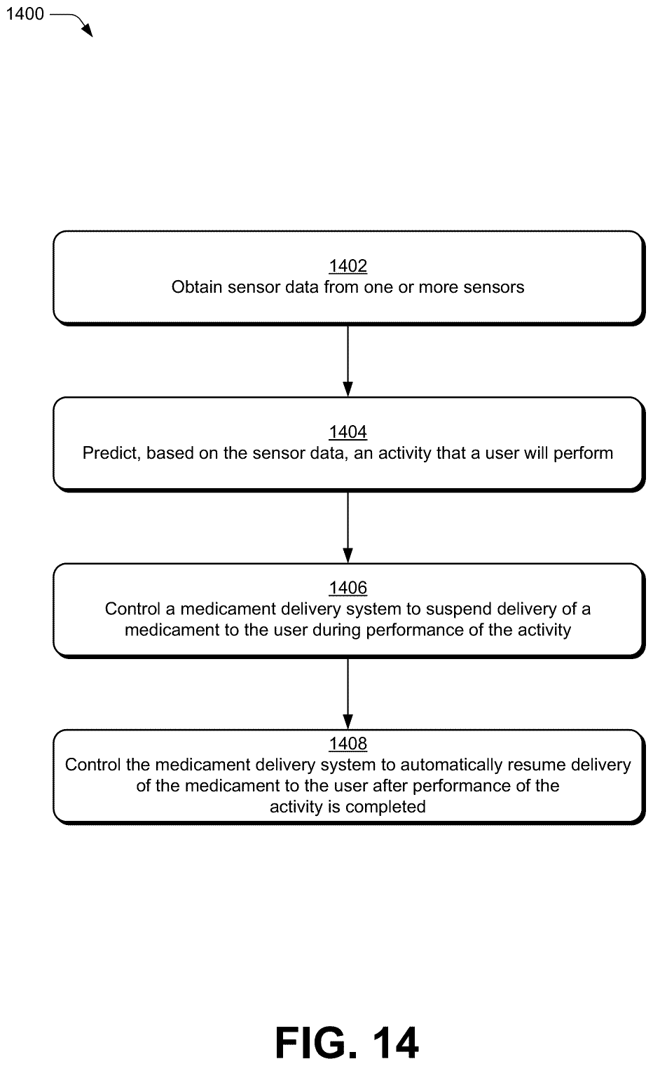

FIG. 14 depicts a procedure 1400 in an example implementation of automatic suspension and resumption of medicament delivery based on an activity.

Sensor data is obtained from one or more sensors (block 1402). By way of example, the delivery device control system 110 obtains sensor data 120. In accordance with the described techniques, the delivery device control system 110 can be configured to receive the sensor data 120 from a variety of different sources 502.

An activity that a user will perform is predicted based on the sensor data (block 1404). By way of example, an activity prediction engine 504 of the delivery device control system 110 predicts an activity 508 that the user will perform based on the sensor data 120. In one or more implementations, the activity 508 is predicted based on sensor data 120 that indicates a location 506 of the user. By way of example, the presence of a user at the gym, within a shower, in bed, and/or sitting in a chair in a workspace, may be significantly informative with respect to the activity 508 being performed. Alternatively or in addition, the activity prediction engine 504 determines the activity 508 of the user based on other sensor data 120, such as based on heart rate data of the user that indicates the user is exercising.

A medicament delivery system is controlled to suspend delivery of a medicament to the user during performance of the activity (block 1406). By way of example, based on the activity 508, the suspend delivery engine 406 of the delivery device control system 110 determines to suspend delivery of a medicament to the user, and outputs the suspend delivery instruction 412 to cause delivery to be suspended. For example, the suspend delivery engine 406 outputs the suspend delivery instruction 412 to the medicament delivery system 106 and the instruction instructs the medicament delivery system 106 to suspend delivery of the medicament to the user.

The medicament delivery system is controlled to automatically resume delivery of the medicament to the user after performance of the activity is completed (block 1408). By way of example, the delivery device control system 110 controls the medicament delivery system 106 to resume delivery of the medicament to the user after performance of the activity 508 is completely. In one or more implementations, the activity prediction engine 504 determines that the activity has been performed by the user, and communicates activity performed instructions to the resume delivery engine 408. The resume delivery engine 408 of the delivery device control system 110 outputs a resume delivery instruction 414 which is communicated to the medicament delivery system 106. Broadly, the resume delivery instruction 414 instructs the medicament delivery system 106 to resume delivery of the medicament.

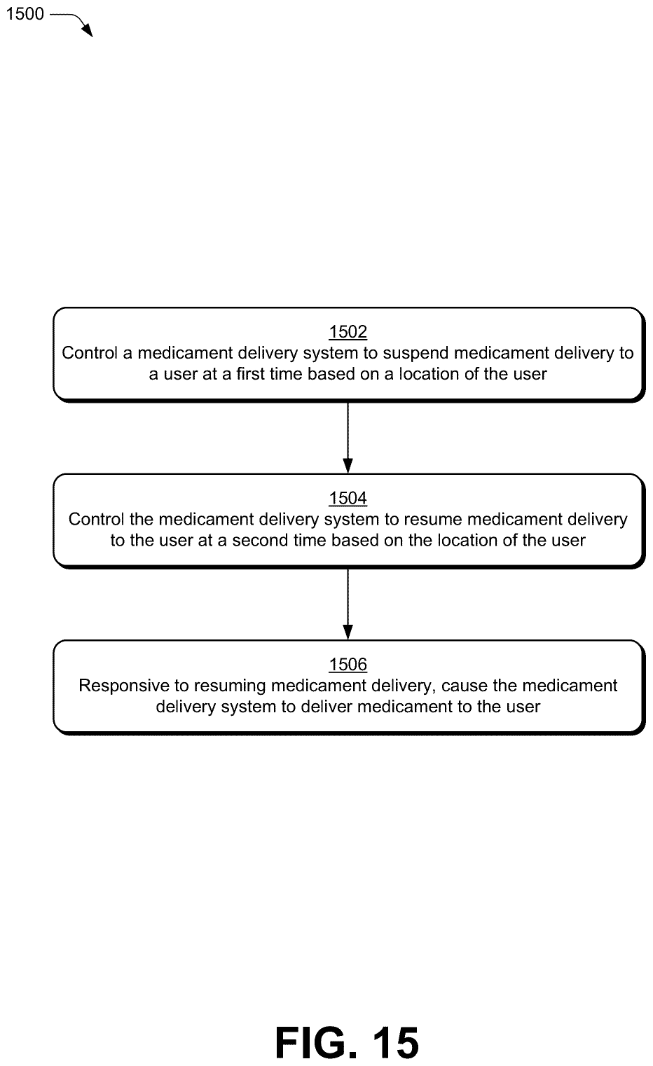

FIG. 15 depicts a procedure 1500 in an example implementation of automatic suspension and resumption of medicament delivery based on a location of the user.

A medicament delivery system is controlled to suspend medicament delivery to a user at a first time based on a location of the user (block 1502). By way of example, the delivery device control system controls the medicament delivery system 106 to suspend medicament delivery to a user at a first time based on a location 506 of the user. As discussed above and below, the location prediction engine 124 predicts the location 506 of the user based on the sensor data 120, which may be obtained from any of a variety of various sources 502. In one or more implementations, based on the location 506, the suspend delivery engine 406 determines to suspend delivery of medicament to the user. In one or more variations, this includes a time at which to suspend the delivery. Further, the suspend delivery engine 406 outputs the suspend delivery instruction 412 to cause delivery to be suspended automatically, e.g., at the time. For example, the suspend delivery engine 406 outputs the suspend delivery instruction 412 to the medicament delivery system 106 and the instruction causes the medicament delivery system 106 to suspend delivery of the medicament to the user. As noted above, the suspend delivery instruction 412 is one example of the instructions 122.

The medicament delivery system is controlled to resume medicament delivery to the user at a second time based on a subsequent location of the user (block 1504). By way of example, the delivery device control system 110 resumes delivery of a medicament automatically based on the subsequent location 602. As discussed above and below, the location prediction engine 124 predicts the subsequent location 602 of the user based on the sensor data 120, which may be obtained from any of a variety of various sources 502. In one or more implementations, the resume delivery engine 408 outputs the resume delivery instruction 414 to cause delivery to be resumed automatically, e.g., at the time. For example, the resume delivery engine 408 outputs the resume delivery instruction 414 to the medicament delivery system 106 and the instruction causes the medicament delivery system 106 to resume delivery of the medicament to the user.

Responsive to resuming medicament delivery, the medicament delivery system is controlled to deliver medicament to the user (block 1506). By way of example, responsive to resuming medicament delivery, the delivery device control system controls the medicament delivery system 106 to deliver the medicament to the user.

Having described examples of procedures in accordance with one or more implementations, consider now an example of a system and device that can be utilized to implement the various techniques described herein.

### Example System and Device

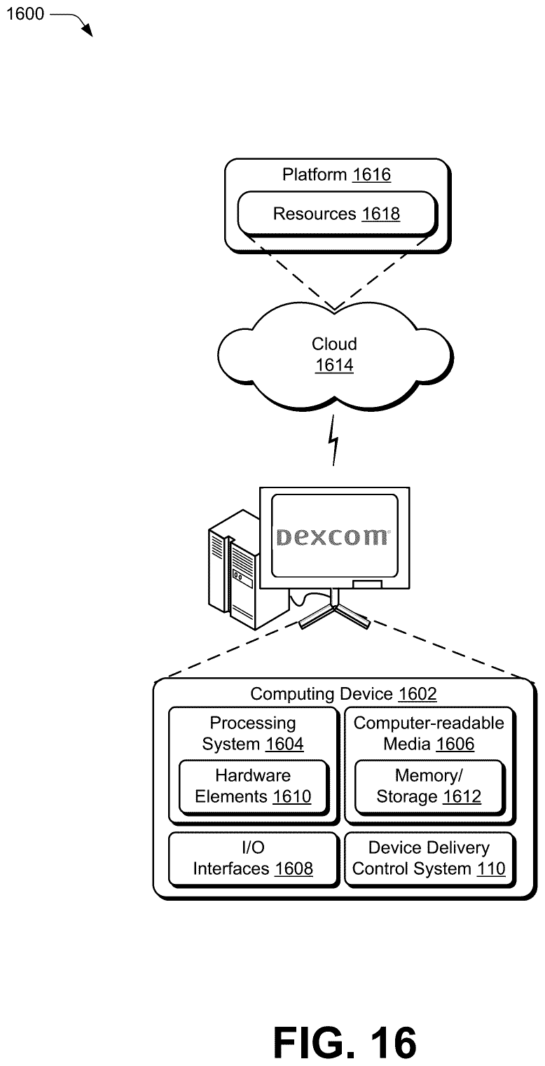

FIG. 16 illustrates an example of a system generally at 1600 that includes an example of a computing device 1602 that is representative of one or more computing systems and/or devices that may implement the various techniques described herein. This is illustrated through inclusion of the delivery device control system 110. The computing device 1602 may be, for example, a server of a service provider, a device associated with a client (e.g., a client device), an on-chip system, and/or any other suitable computing device or computing system.

The example computing device 1602 as illustrated includes a processing system 1604, one or more computer-readable media 1606, and one or more I/O interfaces 1608 that are communicably coupled, one to another. Although not shown, the computing device 1602 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1604 is representative of functionality to perform one or more operations using hardware. Accordingly, the processing system 1604 is illustrated as including hardware elements 1610 that may be configured as processors, functional blocks, and so forth. This may include implementation in hardware as an application specific integrated circuit or other logic device formed using one or more semiconductors. The hardware elements 1610 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 1606 is illustrated as including memory/storage 1612. The memory/storage 1612 represents memory/storage capacity associated with one or more computer-readable media. The memory/storage 1612 may include volatile media (such as random access memory (RAM)) and/or nonvolatile media (such as read only memory (ROM), Flash memory, optical disks, magnetic disks, and so forth). The memory/storage 1612 may include fixed media (e.g., RAM, ROM, a fixed hard drive, and so on) as well as removable media (e.g., Flash memory, a removable hard drive, an optical disc, and so forth). The computer-readable media 1606 may be configured in a variety of other ways as further described below.

Input/output interface(s) 1608 are representative of functionality to allow a user to enter commands and information to computing device 1602, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1602 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 1602. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1602, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1610 and computer-readable media 1606 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1610. The computing device 1602 may be configured to implement particular instructions and/or functions corresponding to the software and/or hardware modules. Accordingly, implementation of a module that is executable by the computing device 1602 as software may be achieved at least partially in hardware, e.g., through use of computer-readable storage media and/or hardware elements 1610 of the processing system 1604. The instructions and/or functions may be executable/operable by one or more articles of manufacture (for example, one or more computing devices 1602 and/or processing systems 1604) to implement techniques, modules, and examples described herein.

The techniques described herein may be supported by various configurations of the computing device 1602 and are not limited to the specific examples of the techniques described herein. This functionality may also be implemented all or in part through use of a distributed system, such as over a “cloud” 1614 via a platform 1616 as described below.

The cloud 1614 includes and/or is representative of a platform 1616 for resources 1618. The platform 1616 abstracts underlying functionality of hardware (e.g., servers) and software resources of the cloud 1614. The resources 1618 may include applications and/or data that can be utilized while computer processing is executed on servers that are remote from the computing device 1602. Resources 1618 can also include services provided over the Internet and/or through a subscriber network, such as a cellular or Wi-Fi network.

The platform 1616 may abstract resources and functions to connect the computing device 1602 with other computing devices. The platform 1616 may also serve to abstract scaling of resources to provide a corresponding level of scale to encountered demand for the resources 1618 that are implemented via the platform 1616. Accordingly, in an interconnected device embodiment, implementation of functionality described herein may be distributed throughout the system 1600. For example, the functionality may be implemented in part on the computing device 1602 as well as via the platform 1616 that abstracts the functionality of the cloud 1614.

### CONCLUSION

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A system comprising:
one or more sensors;
a medicament delivery system configured to deliver a medicament to a user; and
a delivery device control system configured to:
control the medicament delivery system to suspend delivery of the medicament to the user responsive to a request initiated by the user, and to automatically resume delivery of the medicament to the user after a suspension time period has lapsed; and
control the medicament delivery system to automatically suspend delivery of the medicament to the user based on first sensor data obtained from the one or more sensors, and to automatically resume delivery of the medicament to the user based on second sensor data obtained from the one or more sensors.

2. The system of claim 1, wherein the delivery device control system is further configured to control the medicament delivery system to automatically suspend delivery of the medicament to the user based on the first sensor data indicating performance of an activity by the user, and to automatically resume delivery of the medicament to the user based on the second sensor data indicating that performance of the activity is completed.

3. The system of claim 1, wherein the delivery device control system is further configured to control the medicament delivery system to automatically suspend delivery of the medicament to the user based on the first sensor data indicating that the user is at a location, and to automatically resume delivery of the medicament to the user based on second sensor data indicating that the user is at a subsequent location.

4. The system of claim 1, further comprising an analyte monitoring device that is connected to the medicament delivery system to form a closed loop system.

5. The system of claim 4, wherein the analyte monitoring device comprises a wearable glucose monitoring device, and wherein the medicament delivery system comprises a wearable insulin pump.

6. The system of claim 4, wherein the delivery device control system is implemented at a computing device that is wirelessly coupled to the medicament delivery system and the analyte monitoring device.

7. The system of claim 4, wherein the delivery device control system is implemented at the analyte monitoring device.

8. The system of claim 1, wherein the delivery device control system is implemented at the medicament delivery system.

9. A method comprising:
receiving a request to suspend delivery of a medicament to a user;
controlling a medicament delivery system to suspend delivery of the medicament to the user; and
controlling the medicament delivery system to automatically resume medicament delivery to the user after a suspension time period.

10. The method of claim 9, wherein the medicament delivery system automatically resumes medicament delivery to the user after the suspension time period has lapsed without user interaction.

11. The method of claim 9, wherein the medicament delivery system is connected to an analyte monitoring device and a computing device to form a closed loop system.

12. The method of claim 11, wherein the request to suspend delivery of the medicament is received responsive to user input to the medicament delivery system.

13. The method of claim 11, wherein the request to suspend delivery of the medicament is received responsive to user input to a user interface displayed at the computing device.

14. The method of claim 9, wherein the request to suspend delivery of the medicament to the user includes an indication of the suspension time period.

15. The method of claim 9, wherein the suspension time period corresponds to a predetermined period of time.

16. The method of claim 9, wherein the medicament comprises insulin.

17. A method comprising:
obtaining sensor data from one or more sensors;
predicting, based on the sensor data, an activity that a user will perform;
controlling a medicament delivery system to suspend delivery of a medicament to the user during performance of the activity; and
controlling the medicament delivery system to automatically resume delivery of the medicament to the user after performance of the activity is completed.

18. The method of claim 17, further comprising detecting that performance of the activity is completed based on additional sensor data obtained from the one or more sensors.

19. The method of claim 17, wherein the activity comprises exercise, sleeping, or bathing.

20. The method of claim 17, further comprising detecting, based on the sensor data, a location of a user, wherein the predicting further comprises predicting the activity that the user will perform based on the location of the user.

21. The method of claim 20, wherein detecting the location of the user comprises detecting the location of the user based on first sensor data, and confirming the location of the user based on second sensor data.

22. The method of claim 20, wherein detecting the location of the user comprises:
detecting at least two candidate locations of the user based on first sensor data; and
selecting one of the at least two candidate locations as the location of the user based on second sensor data.

23. The method of claim 17, wherein the predicting comprises predicting, based on the sensor data, a time at which the activity will begin.

24. The method of claim 23, further comprising controlling the medicament delivery system to administer a bolus dose of the medicament to the predicted time at which the activity will begin.

25. The method of claim 24, wherein the bolus dose of the medicament comprises a bolus dose of insulin.

26. The method of claim 25, further comprising:
determining a current glucose measurement of the user based on a glucose monitor worn by the user; and
determining the bolus dose of insulin based on the current glucose measurement of the user.

27. A method comprising:
controlling a medicament delivery system to suspend medicament delivery to a user at a first time based on a location of the user;
controlling the medicament delivery system to resume medicament delivery to the user at a second time based on a subsequent location of the user; and
responsive to resuming medicament delivery, causing the medicament delivery system to deliver a dose of medicament to the user.

28. The method of claim 27, wherein the controlling the medicament delivery system to suspend and resume medicament delivery comprises controlling the medicament delivery system to suspend and resume medicament delivery automatically without user input.

29. The method of claim 27, wherein the controlling the medicament delivery system to suspend and resume medicament delivery to the user is not based on analyte data.

30. The method of claim 27, wherein the dose of medicament delivered to the user is based at least in part on analyte data.

31. The method of claim 27, wherein the dose of medicament delivered to the user is based at least in part on a time interval between the first time and the second time.

32. The method of claim 27, wherein the dose of medicament delivered to the user is based at least in part on an activity the user performed between the first time and the second time.

33. The method of claim 32, wherein the activity is predicted based at least in part on the location of the user.
